(19) United States
(12) Patent Application Publication
(10) Pub. No.: US 2009/0170880 A1
(43) Pub. Date: Jul. 2, 2009

(54) Methods and Means for the Treatment of Cancer

(76) Inventor: Olaf Van Tellingen, Zaandam (NL)

Correspondence Address: SWANSON & BRATSCHUN, L.L.C., 8210 SOUTHPARK TERRACE, LITTLETON, CO, 80120, US

(21) Appl. No.: 12/188,028

(22) Filed: Aug. 7, 2008

Related U.S. Application Data
(60) Provisional application No. 61/017,942, filed on Dec. 31, 2007.

(51) Int. Cl. A61K 31/517; A61K 31/4188; A61P 35/04; A61K 31/473

(52) U.S. Cl. 514/266.4; 514/393; 514/297

(57) ABSTRACT

Provided are Breast Cancer Resistance Protein (BCRP) inhibitors, P-glycoprotein (P-gp) inhibitors and chemotherapeutic agents for use in the treatment of cancer by combination therapy of the BCRP inhibitor and/or P-gp inhibitor with the chemotherapeutic agent. The chemotherapeutic agent is an imidazotetrazine, e.g. temozolomide.

### RELATED APPLICATIONS

This application claims the benefit of U.S. Provisional Application Ser. No. 61/017,942, filed Dec. 31, 2007, entitled “Methods and Means for the treatment of Cancer,” which is incorporated herein by reference in its entirety.

### FIELD OF THE INVENTION

The present invention relates to the treatment of cancer using a Breast cancer resistance protein (BCRP) inhibitor and/or a P-glycoprotein (P-gp) inhibitor in combination with a chemotherapeutic agent.

### BACKGROUND TO THE INVENTION

High-grade malignant gliomas such as glioblastoma multiforme (GBM) are refractory to virtually all chemotherapy regimens. Whereas it is possible that the heterogeneity within these tumors favors the presence of innately resistant tumor cells, inadequate drug-exposure of tumor cells because of the blood-brain barrier is most likely a major cause of the general lack of efficacy of chemotherapy.

The blood-brain barrier (BBB) restricts the entry of virtually all commonly used agents (1). Although this barrier is disrupted in the more central parts of the tumor, it is still functional in the peripheral rim that harbors viable and proliferating tumor cells. Moreover, brain tumor cells have the propensity to migrate deep into the surrounding normal brain tissue, where the BBB is still fully intact.

The principal components of the BBB are the endothelial cells that are linked together by complex tight-junctions (2) limiting para-cellular movement of substances. Moreover, transcellular passaging is restricted by the absence of fenestrae and the low endocytic activity of endothelial cells in the brain. Besides these more or less passive restraints, the BBB is also equipped with transport proteins such as P-glycoprotein (P-gp), also known as ABCB1, which is responsible for the limited brain penetration of a wide range of compounds (3). More recently it has been shown that the ABC half-transporter Breast Cancer Resistance Protein (BCRP), also known as ABCG2, also limits the brain penetration of p otentially important substances such as imatinib (4).

For a long time the st andard treatment of GBM consisted of surgery followed by local radiotherapy, with or without nitrosourea-based chemotherapy. A large number of adjuvant nitrosourea-based chemotherapy trials have been performed, but d id not d emonstrate significant survival benefit (5;6)

Recently, temozolomide, an alkylating agent that can be given orally, was found to have significant activity in the treatment of recurrent grade 3 and grade 4 gliomas, with objective response rates of 35% and 8% respectively, and an additional high percentage of disease stabilization (7-9). Progression free survival was significantly longer following temozolomide compared to procarbazine (8). Moreover, a large phase III trial showed a significant survival benefit for rad iotherapy in combination with concomitan t and adjuvant temozolomide compared to radiotherapy alone (10) and this study provided the basis for the new standard treatment of newly diagnosed GBM, where patients start with temozolomide (75 mg/m²/day for 42 days) concomitantly with radiotherapy, subsequently followed by temozolomide monotherapy (150 to 200 mg/m²/day for 5 days with a 23 day rest period). Temozolomide monotherapy remains to be indicated for patients with recurrent GBM or anaplastic astrocytoma.

U.S. Pat. No. 6,703,400 relates to administration of a P-glycoprotein inhibitor in conjunction with an antineoplastic agent. Prados et al. (16) relates to administration of temozolomide in combination with erlotinib.

Glioblastoma multiforme (GBM) is among the deadliest and most devastating of human cancers. The anticancer drug temozolomide is one of the very few drugs that exerts any meaningful response in this disease and has become the mainstay for treatment of patients.

### SUMMARY OF THE INVENTION

Because the brain penetration of temozolomide is apparently already high enough to elicit a meaningful antitumor response, it was not previously thought that entry of temozolomide into the brain might be restricted, e.g. by the blood brain barrier. However, we have surprisingly discovered that brain penetration of temozolomide is limited by P-gp and BCRP, both of which are components of the blood brain barrier.

In particular, by using knockout mouse models we have shown that the brain penetration is increased by 20% when either BCRP or P-gp is absent, and by 50% when both BCRP and P-gp are absent, without affecting the plasma clearance of temozolomide. A greater penetration of temozolomide will lead to a greater concentration of temozolomide in the brain and, therefore, a greater exposure of any cancerous tissue to temozolomide, thereby providing a more effective treatment. This has been confirmed by our finding that administration of temozolomide to P-gp (Mdr1ab) knock-out mice having intracranial tumor xenograft leads to a greater reduction in tumor volume compared to wild-type mice.

This discovery also indicates that BCRP and P-gp will limit the brain penetration of other molecules that are structurally and/or chemically related to temozolomide. In this respect, U.S. Pat. No. 5,260,291 describes imidazotetrazines, including temozolomide, which have antineoplastic activity.

Thus, the invention broadly relates to a method of treating cancer by administering a P-gp inhibitor and/or a BCRP inhibitor in combination with a chemotherapeutic agent, wherein said agent is an imidazotetrazine, such as temozolomide, or a derivative or analogue of temozolomide. This combination therapy will provide an increase in brain concentration of imidazotetrazine, as compared to the brain concentration of imidazotetrazine in the absence of administration of the P-gp and/or BCRP inhibitor and for the same administered dose of imidazotetrazine.

For the avoidance of doubt, in all aspects of this invention, a most preferred “imidazotetrazine” or “imidazotetrazine derivative” is temozolomide.

Our findings indicate that the efficacy of chemotherapy based on temozolomide against brain cancer, such as high grade glioma, can be significantly improved when temozolomide is given in combination with a P-gp/BCRP inhibitor. As temozolomide is one of the very few drugs that exerts any meaningful response against GBM, this invention provides a valuable new treatment.

In a first aspect of the invention there is provided use of a Breast Cancer Resistance Protein (BCRP) inhibitor for the manufacture of a medicament for the treatment of cancer, wherein said medicament is for the treatment of cancer by combination therapy with a P-glycoprotein (P-gp) inhibitor and a chemotherapeutic agent, and wherein the chemotherapeutic agent is an imidazotetrazine.

A medicament containing a BCRP inhibitor may include one or both of a P-gp inhibitor and/or an imidazotetrazine. Alternatively, a medicament containing a BCRP inhibitor may not include one or both of a P-gp inhibitor and/or an imidazotetrazine, which may be provided in the form of further medicaments.

In a further aspect of the invention there is provided use of a P-gp inhibitor for the manufacture of a medicament for the treatment of cancer, wherein said medicament is for the treatment of cancer by combination therapy with a BCRP inhibitor and a chemotherapeutic agent, and wherein the chemotherapeutic agent is an imidazotetrazine.

A medicament containing a P-gp inhibitor may include one or both of a BCRP inhibitor and/or an imidazotetrazine. Alternatively, a medicament containing a P-gp inhibitor may not include one or both of a BCRP inhibitor and/or an imidazotetrazine, which may be provided in the form of further medicaments.

In a further aspect of the invention there is provided use of a chemotherapeutic agent for the manufacture of a medicament for the treatment of cancer, wherein said medicament is for the treatment of cancer by combination therapy with a BCRP inhibitor and a P-gp inhibitor, and wherein the chemotherapeutic agent is an imidazotetrazine.

A medicament containing a chemotherapeutic agent may include one or both of a BCRP inhibitor and/or a P-gp inhibitor. Alternatively, a medicament containing a chemotherapeutic agent may not include one or both of a BCRP inhibitor and/or a P-gp inhibitor, which may be provided in the form of further medicaments.

In a further aspect of the invention there is provided a BCRP inhibitor for use in the treatment of cancer by combination therapy of said BCRP inhibitor with a P-gp inhibitor and a chemotherapeutic agent, wherein the chemotherapeutic agent is an imidazotetrazine.

In a further aspect of the invention there is provided a P-gp inhibitor for use in the treatment of cancer by combination therapy of said P-gp inhibitor with a BCRP inhibitor and a chemotherapeutic agent, wherein the chemotherapeutic agent is an imidazotetrazine.

In a further aspect of the invention there is provided a chemotherapeutic agent for use in the treatment of cancer by combination therapy of said chemotherapeutic agent with a BCRP inhibitor and a P-gp inhibitor, wherein the chemotherapeutic agent is an imidazotetrazine.

Combination therapy of a BCRP inhibitor, a P-gp inhibitor and imidazotetrazine may involve simultaneous, separate (chronologically staggered) or sequential administration of the BCRP inhibitor, the P-gp inhibitor and the imidazotetrazine. Preferably the therapeutic effect and/or brain concentration of imidazotetraazine provided by the combination therapy is greater than the therapeutic effect and/or brain concentration of imidazotetrazine provided by administration of an equivalent dose of the imidazotetrazine in the absence of the BCRP inhibitor and P-gp inhibitor.

Thus, in a further aspect of the invention there are provided products containing a BCRP inhibitor, a P-gp inhibitor and a chemotherapeutic agent, wherein the chemotherapeutic agent is an imidazotetrazine, as a combined preparation for simultaneous, separate or sequential use in cancer therapy.

In a further aspect of the invention there is provided a pharmaceutical composition comprising a BCRP inhibitor, a P-gp inhibitor and a chemotherapeutic agent, wherein the chemotherapeutic agent is an imidazotetrazine. Preferably the pharmaceutical composition is for treating cancer in a patient.

In a further aspect of the invention there is provided a kit of parts comprising packaging and/or a container containing a BCRP inhibitor, a P-gp inhibitor, and a chemotherapeutic agent, wherein the chemotherapeutic agent is an imidazotetrazine. Preferably the kit is for treating cancer in a patient. Preferably the kit also includes instructions for the administration of the BCRP inhibitor, the P-gp inhibitor and the chemotherapeutic agent in a patient in need of treatment in order to treat cancer. The BCRP inhibitor, the P-gp inhibitor, and the chemotherapeutic agent may each be present in separate containers, or at least two of these, or all three, may be present in the same container.

In a further aspect of the invention there is provided a method comprising administering a BCRP inhibitor, a P-gp inhibitor and a chemotherapeutic agent to an animal or patient, wherein the chemotherapeutic agent is an imidazotetrazine. Preferably the method is a method of treating a patient, and is more preferably a method of treating cancer in a patient.

The BCRP inhibitor, P-gp inhibitor and imidazotetrazine may be administered simultaneously, separately (chronologically staggered) or sequentially. Preferably the therapeutic effect and/or brain concentration of imidazotetrazine provided by administration of the BCRP, the P-gp inhibitor and the imidazotetrazine is greater than the therapeutic effect and/or brain concentration of imidazotetrazine provided by administration of an equivalent dose of the imidazotetrazine in the absence of the BCRP inhibitor and the P-gp inhibitor.

As mentioned above, we have surprisingly discovered that BCRP and P-gp limit penetration of temozolomide in the brain.

Thus, the BCRP inhibitor is preferably for inhibiting, e.g. antagonising, BCRP, particularly BCRP in the blood brain barrier. The cells comprising the BCRP to be inhibited by BCRP inhibitors may be normal, i.e. non-cancerous cells.

Preferably, the BCRP inhibitor is for increasing the penetration of an imidazotetrazine, such as temozolomide, into the CNS of a patient, e.g. the brain tissue, compared to the penetration of the imidazotetrazine in the absence of the BCRP inhibitor for the same dose of the imidazotetrazine. Preferably the BCRP inhibitor is for increasing the amount, e.g. the concentration, of the imidazotetrazine in the CNS of a patient, e.g. the brain tissue, compared to the amount of the imidazotetrazine in the CNS in the absence of the BCRP inhibitor for the same dose of the imidazotetrazine. Preferably the BCRP inhibitor is for exposing the CNS of a patient, e.g. the brain tissue, to the imidazotetrazine for a longer period of time compared to the period of time that the CNS is exposed to the imidazotetrazine in the absence of the BCRP inhibitor for the same dose of the imidazotetrazine.

Furthermore, the BCRP inhibitor is preferably for inhibiting, e.g. antagonising, the transport activity of BCRP, particularly the blood brain barrier transport activity mediated by BCRP. Preferably the BCRP inhibitor is for reducing transport of an imidazotetrazine across the blood brain barrier of a patient, particularly from the central nervous system (CNS) to the vasculature, such as from the brain to the blood, i.e. the brain capillary bed. Said reduction in transport is preferably as compared to the transport of the imidazotetrazine across the blood brain barrier in the absence of the BCRP inhibitor for the same dose of the imidazotetrazine.

Likewise, the P-gp inhibitor is preferably for inhibiting, e.g. antagonising, P-gp, particularly P-gp in the blood brain barrier. The cells comprising the P-gp to be inhibited by P-gp inhibitors may be normal, i.e. non-cancerous cells.

Preferably, the P-gp inhibitor is for increasing the penetration of an imidazotetrazine, such as temozolomide, into the CNS of a patient, e.g. the brain tissue, compared to the penetration of the imidazotetrazine in the absence of the P-gp inhibitor for the same dose of the imidazotetrazine. Preferably the P-gp inhibitor is for increasing the amount, e.g. the concentration, of the imidazotetrazine in the CNS of a patient, e.g. the brain tissue, compared to the amount of the imidazotetrazine in the CNS in the absence of the P-gp inhibitor for the same dose of the imidazotetrazine. Preferably the P-gp inhibitor is for exposing the CNS of a patient, e.g. the brain tissue, to the imidazotetrazine for a longer period of time compared to the period of time that the CNS is exposed to the imidazotetrazine in the absence of the BCRP inhibitor for the same dose of the imidazotetrazine.

Furthermore, the P-gp inhibitor is preferably for inhibiting, e.g. antagonising, the transport activity of P-gp, particularly the blood brain barrier transport activity mediated by P-gp. Preferably the P-gp inhibitor is for reducing transport of an imidazotetrazine across the blood brain barrier of a patient, particularly from the central nervous system (CNS) to the vasculature, such as from the brain to the blood, i.e. the brain capillary bed. Said reduction in transport is preferably as compared to the transport of the imidazotetrazine across the blood brain barrier in the absence of the P-gp inhibitor for the same dose of the imidazotetrazine.

Likewise, the method of treating a patient preferably comprises causing an increase in the amount, e.g. the concentration, of the imidazotetrazine in the brain of a patient, compared to the amount of the imidazotetrazine in the brain tissue of the patient when administered with the same dose of the imidazotetrazine in the absence of the BCRP inhibitor and/or the P-gp inhibitor. Preferably the method of treating a patient comprises causing a reduction, e.g. inhibition of, the transport of the imidazotetrazine across the blood brain barrier, e.g. compared to the transport of the imidazotetrazine across the blood brain barrier of the patient when administered with the same dose of the imidazotetrazine in the absence of the BCRP inhibitor and/or the P-gp inhibitor.

In a further aspect of the invention there is provided use of a BCRP inhibitor for the manufacture of a medicament for the treatment of cancer by combination therapy with a chemotherapeutic agent, wherein the BCRP inhibitor is for increasing the amount of the chemotherapeutic agent in the brain of a patient being treated, and wherein the chemotherapeutic agent is an imidazotetrazine. A medicament containing a BCRP inhibitor may or may not include an imidazotetrazine.

In a further aspect of the invention there is provided use of a P-gp inhibitor for the manufacture of a medicament for the treatment of cancer by combination therapy with a chemotherapeutic agent, wherein the P-gp inhibitor is for increasing the amount of the chemotherapeutic agent in the brain of a patient being treated, and wherein the chemotherapeutic agent is an imidazotetrazine. A medicament containing a P-gp inhibitor may or may not include an imidazotetrazine.

In a further aspect of the invention there is provided a BCRP inhibitor for use in the treatment of cancer by combination therapy of said BCRP inhibitor with a chemotherapeutic agent, wherein the BCRP inhibitor is for increasing the amount of the chemotherapeutic agent in the brain of a patient being treated, and wherein the chemotherapeutic agent is an imidazotetrazine. For example, the BCRP inhibitor may be for use in, or may be used for, increasing the amount of the chemotherapeutic agent in the brain of the patient being treated.

In a further aspect of the invention there is provided a P-gp inhibitor for use in the treatment of cancer by combination therapy of said P-gp inhibitor with a chemotherapeutic agent, wherein the P-gp inhibitor is for increasing the amount of the chemotherapeutic agent in the brain of a patient being treated, and wherein the chemotherapeutic agent is an imidazotetrazine. For example, the P-gp inhibitor may be for use in, or may be used for, increasing the amount of the chemotherapeutic agent in the brain of the patient being treated.

Combination therapy of a BCRP inhibitor and an imidazotetrazine or combination therapy of a P-gp inhibitor and an imidazotetrazine may involve simultaneous, separate (chronologically staggered) or sequential administration of the BCRP inhibitor and the imidazotetrazine or the P-gp inhibitor and the imidazotetrazine, respectively. Preferably the therapeutic effect and/or brain concentration of imidazotetrazine provided by the combination therapy is greater than the therapeutic effect and/or brain concentration of imidazotetrazine provided by administration of an equivalent dose of the imidazotetrazine in the absence of the BCRP inhibitor or the P-gp inhibitor.

Thus, in a further aspect of the invention there are provided products containing a BCRP inhibitor and a chemotherapeutic agent, as a combined preparation for simultaneous, separate or sequential use in cancer therapy, wherein the BCRP inhibitor is for increasing the amount of the chemotherapeutic agent in the brain of a patient being treated, and wherein the chemotherapeutic agent is an imidazotetrazine.

In a further aspect of the invention there are provided products containing a P-gp inhibitor and a chemotherapeutic agent, as a combined preparation for simultaneous, separate or sequential use in cancer therapy, wherein the P-gp inhibitor is for increasing the amount of the chemotherapeutic agent in the brain of a patient being treated, and wherein the chemotherapeutic agent is an imidazotetrazine.

In a further aspect of the invention there is provided a pharmaceutical composition comprising a BCRP inhibitor and a chemotherapeutic agent, wherein the BCRP inhibitor is for increasing the amount of the chemotherapeutic agent in the brain of a patient, and wherein the chemotherapeutic agent is an imidazotetrazine.

In a further aspect of the invention there is provided a pharmaceutical composition comprising a P-gp inhibitor and a chemotherapeutic agent, wherein the pharmaceutical composition is for use in the treatment of cancer, wherein the P-gp inhibitor is for increasing the amount of the chemotherapeutic agent in the brain of a patient being treated, and wherein the chemotherapeutic agent is an imidazotetrazine.

In a further aspect of the invention there is provided a kit of parts comprising a packaging and/or a container containing a BCRP inhibitor and a chemotherapeutic agent, wherein the kit is for use in the treatment of cancer, wherein the BCRP inhibitor is for increasing the amount of the chemotherapeutic agent in the brain of a patient being treated, and wherein the chemotherapeutic agent is an imidazotetrazine. Preferably the kit also includes instructions for the administration of the BCRP inhibitor and the chemotherapeutic agent in a patient in need of treatment in order to treat cancer. The BCRP inhibitor and the chemotherapeutic agent may be in the same or separate containers.

In a further aspect of the invention there is provided a kit of parts comprising a packaging and/or a container containing a P-gp inhibitor and a chemotherapeutic agent, wherein the kit is for use in the treatment of cancer, wherein the P-gp inhibitor is for increasing the amount of the chemotherapeutic agent in the brain of a patient being treated, and wherein the chemotherapeutic agent is an imidazotetrazine. Preferably the kit also includes instructions for the administration of the P-gp inhibitor and the chemotherapeutic agent in a patient in need of treatment in order to treat cancer. The P-gp inhibitor and the chemotherapeutic agent may be in the same or separate containers.

Preferably the BCRP inhibitor and/or the P-gp inhibitor is for increasing the amount of the imidazotetrazine in the brain of the patient compared to the amount of the imidazotetrazine in the brain of the patient in the absence of the BCRP inhibitor and/or the P-gp inhibitor for the same dose of the imidazotetrazine. Preferably the BCRP inhibitor and/or the P-gp inhibitor is for exposing the CNS of a patient, e.g. the brain tissue, to the imidazotetrazine for a longer period of time compared to the period of time that the CNS is exposed to the imidazotetrazine in the absence of the BCRP inhibitor for the same dose of the imidazotetrazine. Preferably the BCRP inhibitor and/or the P-gp inhibitor is for reducing transport of the imidazotetrazine across the blood brain barrier, e.g. from the brain to the blood, of the patient being treated, e.g. compared to the transport of the imidazotetrazine across the blood brain barrier in the absence of the BCRP inhibitor and/or the P-gp inhibitor for the same dose of the imidazotetrazine.

In a further aspect of the invention there is provided a method of treating a patient, said method comprising administering a BCRP inhibitor and a chemotherapeutic agent to the patient, wherein administration of the BCRP inhibitor causes an increase in the amount of the chemotherapeutic agent in the brain of the patient being treated, and wherein the chemotherapeutic agent is an imidazotetrazine. Preferably the method of treating a patient is a method of treating cancer in a patient.

In a further aspect of the invention there is provided a method of treating a patient, said method comprising administering a P-gp inhibitor and a chemotherapeutic agent to the patient, wherein administration of the P-gp inhibitor causes an increase in the amount of the chemotherapeutic agent in the brain of the patient being treated, and wherein the chemotherapeutic agent is an imidazotetrazine. Preferably the method of treating a patient is a method of treating cancer in a patient.

Preferably the increase in the amount of the imidazotetrazine in the brain of the patient is an increase compared to the amount of the imidazotetrazine in the brain of the patient in the absence of the BCRP inhibitor and/or the P-gp inhibitor for the same dose of the imidazotetrazine. Preferably the method of treating a patient comprises exposing the CNS, e.g. the brain tissue of a patient to the imidazotetrazine for a longer period of time compared to the period of time that the CNS is exposed to the imidazotetrazine in the absence of the BCRP inhibitor and/or the P-gp inhibitor for the same dose of the imidazotetrazine. Preferably the method of treating a patient comprises causing a reduction in the transport of the imidazotetrazine across the blood brain barrier, e.g. from the brain to the blood, of the patient, e.g. a reduction compared to the amount of the imidazotetrazine in the brain of the patient in the absence of the BCRP inhibitor and/or the P-gp inhibitor for the same dose of the imidazotetrazine.

The BCRP inhibitor and the imidazotetrazine or the P-gp inhibitor and the imidazotetrazine may be administered simultaneously, separately (chronologically staggered) or sequentially. Preferably the therapeutic effect and/or brain concentration of imidazotetrazine provided by the administration of the BCRP inhibitor and/or the P-gp inhibitor, and imidazotetrazine is greater than the therapeutic effect and/or brain concentration of imidazotetrazine provided by administration of an equivalent dose of the imidazotetrazine in the absence of the BCRP inhibitor and/or the P-gp inhibitor.

The present invention also provides the following aspects and preferred embodiments:

An imidazotetrazine chemotherapeutic agent for use in a method of treatment of cancer of the central nervous system in combination with elacridar. More preferably, temozolomide for use in a method of treatment of cancer of the central nervous system in combination with elacridar.

Use of an imidazotetrazine chemotherapeutic agent in the manufacture of a medicament for treatment of a cancer of the central nervous system in combination with elacridar is also provided.

Elacridar for use in a method of treatment of cancer of the central nervous system in combination with an imidazotetrazine chemotherapeutic agent. More preferably, elacridar for use in a method of treatment of cancer of the central nervous system in combination with temozolomide.

Use of elacridar in the manufacture of a medicament for treatment of a cancer of the central nervous system in combination with temozolomide is also provided.

A method of treating a patient having cancer of the central nervous system, the method comprising administering to the patient a therapeutically effective amount of elacridar, and a therapeutically effective amount of temozolomide.

The invention includes the combination of the aspects and preferred features described, including preferred features described below, except where such a combination is clearly impermissible or expressly avoided.

Aspects and embodiments of the present invention will now be illustrated, by way of example, with reference to the apparent to those skilled in the art. All documents mentioned in this text are incorporated herein by reference.

### BRIEF DESCRIPTION OF THE FIGURES

Embodiments and experiments illustrating the principles of the invention will now be discussed with reference to the accompanying figures in which:

FIG. 1. Transwell experiments with temozolomide. Temozolomide was added to the basolateral or apical compartment of the transwell to measure basolateral-to-apical (b>a) (filled circles) or apical-to-basolateral (a>b) (open circles) transport, respectively, using LLC-PK1 (parent) versus Mdr1a (mouse) and MDR1 (human) transduced sublines or using MDCKII (parent) versus Bcrp1 (mouse) and BCRP (human) transduced sublines. Because temozolomide is unstable at pH 7.4 (about 50% degradation in 30 min) half the volume of the donor compartment was replaced by a freshly prepared drug solution every 30 min. The transport of temozolomide is depicted as percentage of temozolomide initially present at the donor compartment. Because degradation will also take place in the acceptor compartment during the experiment, this value will be an underestimation of the fraction that is actually translocated. Temozolomide transport by Mdr1a/MDR1 was not readily detected by this assay. Transport by Bcrp1/BCRP was evident; transport by the human BCRP transduced is less because the relatively poor quality of expression of BCRP in this cell line. (Putative) inhibitors of Bcrp1 were added at the depicted concentrations. (a) LLC-PK1 cells, (b) LLC-Mdr1a cells, (c) LLC-MDR1 cells, (d) MDCK parent cells, (e) MDCKII-Bcrp1 cells, (f) MDCK-BCRP cells, (g) MDCKII-Bcrp1 cells+5 μM GF120918 (elacridar), (h) MDCKII-Bcrp1 cells+5 μM erlotinib, (i) MDCKII-Bcrp1 cells+10 μM erlotinib, (j) MDCKII-Bcrp1 cells+10 μM gefitinib, (k) MDCKII-Bcrp1 cells+10 μM Novobiocin, (l) MDCKII-Bcrp1 cells+50 μM Novobiocin.

FIG. 2. Brain concentration (μg/g) (a) and plasma concentration (μg/ml) (b) of temozolomide in mice. Wild-type (WT), Bcrp1, Mdr1ab and Bcrp1/Mdr1ab knockout (KO) mice received 50 mg/kg of temozolomide by i.v. injection. Cohorts of mice were killed at several time points between 15 min and 7 h. Temozolomide levels in plasma and brain homogenates were analyzed by high-performance liquid chromatography.

FIG. 3. The efficacy of temozolomide against intracranial Mel57-luc (luciferase transfected subline) tumor xenografts in wild-type nude mice (WT) and mdrlab knockout nude mice (MDR). Animals received temozolomide per os (oral) 100 mg/kg for 5 consecutive days starting November 27. Tumor volume was measured by bioluminescence activity.

FIG. 4. Chemical structures of elacridar (I), [3H]-imidazo[5,1-d]-1,2,3,5-tetrazin-4-one (II) in which R¹ and R² are as defined herein, and temozolomide (III).

FIG. 5. Brain and plasma concentration of temozolomide at 2 h after drug administration with or without elacridar (upper) or gefitinib (lower panel). Elacridar enhanced the brain penetration (brain-to-plasma ratio) significantly in wild-type mice (p=0.001) to levels that were similar as in Bcrp1/Mdr1ab knockout mice. The difference in temozolomide brain-to-plasma ratio between Bcrp1/Mdr1ab knockout mice with or without elacridar was not significantly different.

FIG. 6. Brain and plasma concentration of temozolomide at 2 h after drug administration with or without gefitinib. Gefitinib did not enhance the brain penetration of temozolomide in wild-type mice, but significantly increased the plasma level (p=0.001). The difference in brain levels found in Bcrp1/Mdr1ab knockout mice without or with erlotinib is most likely due to the higher plasma level.

FIG. 7. Plasma concentration-time curves of temozolomide after oral administration. Temozolomide (100 mg/kg) was given orally to wild-type (n=18) and Bcrp1/Mdr1ab knockout mice (n=19) and serial blood samples were drawn from the tail vein. Depicted is the curve of the main±SE concentrations.

FIG. 8. Efficacy of temozolomide against an intracranial tumor implanted in wild-type and Bcrp1/Mdr1ab deficient nude mice. Mel57-luc cells were injected stereotactically and monitored using in vivo bioluminescence imaging on an IVIS200 camera. Treatment with temozolomide (100 mg/kg/day×5) was started at day 7 following tumor cell injection. Mel57-luc cells do not disrupt the blood-brain barrier when growing inside the brain parenchyma.

FIG. 9. Efficacy of temozolomide with GF120918 (elacridar) against an intracranial tumor implanted in wild-type nude mice. Mel57-luc cells were injected stereotactically and treatment with oral temozolomide (100 mg/kg/day×5) given 20-30 min after oral elacridar (GF120918, 100 mg/kg/day×5) was started at day 7 following tumor cell injection. The combination of elacridar and temozolomide is shown to be more effective than temozolomide alone against Mel57 tumors in wild-type (P-gp/Bcrp proficient) mice.

### DETAILED DESCRIPTION OF THE INVENTION

BCRP and P-gp

“Breast Cancer Resistance Protein” (BCRP) is also known as ABCG2 and preferably refers to the human BCRP or the murine BCRP. More preferably the term refers to human BCRP.

Human BCRP is also known as ATP-binding cassette sub-family G member 2, Placenta-specific ATP-binding cassette transporter, Mitoxantrone resistance-associated protein, and CDw338 antigen, and the NCBI (http://www.ncbi.nlm.nih.gov/) database accession number for the amino acid sequence is Q9UNQ0 (GI:67462103). Murine BCRP is also known as ATP-binding cassette sub-family G member 2 and Breast cancer resistance protein 1 homolog, and the NCBI database accession number for the amino acid sequence is Q7TMS5 (GI:68052328).

“P-glycoprotein” (P-gp) is also known as ABCB1, and preferably refers to the human P-gp or the murine P-gp. There are two murine P-gp proteins, Mdr1a and Mdr1b, but only Mdr1a P-glycoprotein is expressed in the mouse brain. Thus when the term refers to murine P-gp the P-gp is preferably Mdr1a. More preferably the term refers to human P-gp.

Human P-gp is also known as Multidrug resistance protein 1 (EC) and P-glycoprotein 1, and the NCBI database accession number for the amino acid sequence is P08183 (GI:2506118). Murine P-gp MDR1A is also known as Multidrug resistance protein 3 (EC) and P-glycoprotein 3, and the NCBI database accession number for the amino acid sequence is P21447 (GI:266517). Murine P-gp MDR1B is also known as Multidrug resistance protein 1 (EC) and P-glycoprotein 1, and the NCBI database accession number for the amino acid sequence is P06795 (GI:126927).

Amino acid sequences and/or nucleotide sequences are available from public databases, such as http://harvester.embl.de/ and http://www.ncbi.nlm.nih.gov/.

BCRP Inhibitors and P-gp Inhibitors

A BCRP inhibitor is any entity that inhibits, e.g. antagonises, the activity of BCRP such that the ability of BCRP to perform its normal biological function is reduced. A BCRP inhibitor may inhibit BCRP by binding, e.g. selectively (specifically) binding, to BCRP. For example, binding of a BCRP inhibitor to BCRP may prevent temozolomide from docking with BCRP, e.g. by blocking the docking site or by changing the configuration of the docking site by an allosteric interaction, thereby preventing BCRP from transporting temozolomide, e.g. across a membrane.

Likewise, a P-gp inhibitor is any entity that inhibits, e.g. antagonises, the activity of P-gp such that the P-gp inhibitor reduces the ability of P-gp to perform its normal biological function. A P-gp inhibitor may inhibit P-gp by binding, e.g. selectively (specifically) binding, to P-gp. For example, binding of a P-gp inhibitor to P-gp may prevent temozolomide from docking with P-gp, e.g. by blocking the docking site or by changing the configuration of the docking site by an allosteric interaction, thereby preventing BCRP from transporting temozolomide e.g. across a membrane.

Preferably a BCRP inhibitor or a P-gp inhibitor has a low IC50 (concentration of the inhibitor required for 50% inhibition) for BCRP or P-gp. Preferably the IC50 is in the μM or nM range. Preferably a BCRP inhibitor or a P-gp inhibitor has an IC50 of less than 5000 nM, 3000 nM, 2000 nM, 1000 nM, 800 nM, 500 nM, 400 nM, 300 nM, 200 nM or 100 nM. The IC50 may be between 10 nM and 5000 nM, more preferably between 10 nM and 1000 nM, still more preferably between 10 nM and 500 nM or between 10 nM and 200 nM.

Preferably a BCRP inhibitor or a P-gp inhibitor has a low Kᵢ for BCRP or P-gp, e.g. in the μM or nM range. Preferably, a BCRP inhibitor or a P-gp inhibitor has a Kᵢ of less than 50 μM, more preferably less than 40 μM, 30 μM, 20 μM or 10 μM. The Kᵢ may be between 10 nM and 50 μM, more preferably between 10 nM and 30 μM, still more preferably between 10 nM and 20 μM or between 10 nM and 10 μM.

Binding affinity (Kᵢ) can be calculated from the IC₅₀ using the equation of Cheng and Prusoff (Cheng, Y., Prusoff, W. H. (1973) Biochem. Pharmacol. 22, 3099-3108):

Kᵢ=IC₅₀÷{1+([Radioligand]/K_{d})}

The same entity may be a BCRP inhibitor and also a P-gp inhibitor, i.e. a single agent may exhibit BCRP inhibitory activity and P-gp inhibitory activity. Examples of such agents are elacridar and XR9576 (tariquidar).

A BCRP inhibitor or a P-gp inhibitor may be, or may comprise, an organic compound, such as a small organic molecule; a peptide; a protein; an antibody (e.g. a polyclonal antibody, monoclonal antibody, single chain antibody, CDR-grafted antibody, humanised antibody); or an aptamer. A BCRP inhibitor or a P-gp inhibitor may be, or may comprise, one molecule or may comprise more than one molecule, e.g. it may be an entity which comprises molecules that are connected by bonds other than covalent bonds, such as ionic bonds and/or van der Waals interactions.

Alternatively, a BCRP or P-gp inhibitor may be an antisense inhibitor, e.g. an oligonucleotide that down-regulates cellular expression of BCRP or P-gp. According to the present invention, down-regulation of BCRP and/or P-gp expression in the cells of the blood brain barrier will reduce the transport of imidazotetrazines across the blood brain barrier. U.S. Pat. No. 6,001,991 and U.S. Pat. No. 5,866,699 relate to down-regulation of the expression of P-gp using antisense oligonucleotides.

A BCRP inhibitor or a P-gp inhibitor may be a prodrug, e.g. an entity that does not exhibit inhibitory activity, but is converted by physiological processes in the body of a patient into a different entity that does exhibit inhibitory activity.

Examples of BCRP inhibitors and P-gp inhibitors are disclosed in WO 2006/012958 and WO 00/69390. Examples of BCRP inhibitors are also disclosed in Mao Q, Unadkat J D, AAPS Journal 2005 7(1): E118-E133; Schlegel et al. Abstract 4415, Proc. Am. Ass. Cancer Res. Vol. 40, 1999, page 669; and Rabindran et al., Abstract 2093, ibid. page 315.

Preferably a BCRP inhibitor is selected from the group consisting of: erlotinib, pantoprazole, the Aspergillus fumigatus secondary metabolite tryprostatin A, fumitremorgin C abbreviated as FTC and its derivatives the demethoxy-fumitremorgin C analogs, Kol32, Kol34, Kol43, GF120918 (elacridar), the quinazoline-based HER family tyrosine kinase inhibitor CI1033, estrogens like estrone and 17beta-estradiol, e.g. estradiol-17-beta-D-glucuronide, iressa (gefitinib or ZD1839), imatinib mesylate (STI571 or Gleevec), EKI-785, Novobiocin, diethylstilbestrol, Tamoxifen, TAG-11, TAG0139, reserpine, VX710 (Biricodar or Incel), Tryprostatin A, Flavonoids (chrysin and biochanin A), Ritonavir, Saquinavir, Nelfinavir, Omeprazole, Cyclosporine A, and XR9051 and XR9576 (tariquidar) from Xenova. The following categories of compounds can be considered to provide BCRP inhibitors of varying specificity: acridine derivatives, quinoline derivatives, and isoquinoline derivatives. Preferably the BCRP inhibitor is elacridar. Optionally the BCRP is not erlotinib.

Preferably a P-gp inhibitor is selected from the group consisting of: cyclosporine A; verapamil, [3′-desoxy-3′-oxo-MeBmt]¹-Ciclosporin, [3′-desoxy-3′-oxo-MeBmt]¹-[Val]²-Ciclosporin (also known as valspodar or PSC388 and which can be administered in the form of the galenical composition as disclosed in WO 93/20833) and [3′-desoxy-3′-oxo-MeBmt]¹-[Nva]²-Ciclosporin disclosed in EP 0 296 122 in Example H as cyclosporins 1.37, 1.38 and 1.39, respectively; Cyclo-[Pec-MeVal-Val-MeAsp(β-O-t-Bu)-MeIle-MeIle-Gly-MeVal-Tyr(Me)-L-Lact] and Cyclo-[Pec-MeVal-Val-MeAsp-MeIle-MeIle-Gly-MeVal-Tyr(Me)-D-Lact], disclosed in EP 0 360 760 as Examples 52 and 1 (first compound), respectively; GF120918 (elacridar), MS-209, XR-9576, VX-710, R-101933, NSC-38721, OC-144093, and LY-335979, disclosed in Liscovitch and Lavie, 2002, Vol. 5, 349-55, and XR9051 and XR9576 (tariquidar) from Xenova. Optionally the P-gp inhibitor is not SCH66336 as disclosed in U.S. Pat. No. 6,703,400.

The BCRP inhibitors and P-gp inhibitors also include pharmaceutically acceptable salts, esters and hydrates, or may be crystallised with other solvents used for crystallisation.

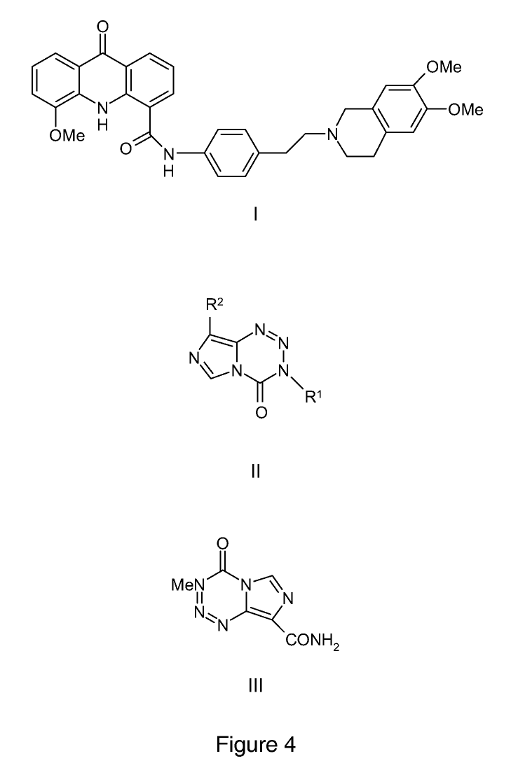

Preferably the BCRP inhibitor and/or the P-gp inhibitor is GF120918 (elacridar), which has the chemical formula I (FIG. 4).

Analogues and derivatives of the above compounds may also be BCRP inhibitors or P-gp inhibitors. Such analogues and/or derivatives may be identified as BCRP and/or P-gp inhibitors according to the methods described below.

Imidazotetrazines

Imidazotetrazines according to the present invention include those disclosed in U.S. Pat. No. 5,260,291. According to U.S. Pat. No. 5,260,291 these compounds possess valuable antineoplastic activity and are therefore chemotherapeutic agents according to the present invention.

Thus, an imidazotetrazine is preferably a [3H]-imidazo[5,1-d]-1,2,3,5-tetrazin-4-one derivative having the formula II (FIG. 4).

In Formula II R² represents a carbamoyl group, or a carbamoyl group in which the nitrogen atom is bound to one or two groups selected from alkyl and alkenyl containing up to 4 carbon atoms, and cycloalkyl groups containing from 3 to 8 carbon atoms, and, when R¹ represents hydrogen, alkali metal salts thereof.

In addition, R¹ represents hydrogen, or an alkyl, alkenyl or alkynyl group containing from 1 to 6 carbon atoms, or a said group substituted by from 1 to 3 substituents selected from halogen atoms, alkoxy, alkylthio, alkylsulphinyl and alkylsulphonyl groups containing up to 4 carbon atoms, and phenyl groups substituted by alkoxy and alkyl groups containing from 1 to 4 carbon atoms or a nitro group. Alternatively, R¹ represents a cycloalkyl group containing from 3 to 8 carbon atoms.

The preparation of the above [3H]-imidazo[5,1-d]-1,2,3,5-tetrazin-4-one derivatives is described in U.S. Pat. No. 5,260,291.

Preferably the imidazotetrazine is chosen from the group consisting of:

8-carbamoyl-3-methyl-[3H]-imidazo[5,1-d]-1,2,3,5-tetrazin-4-one;

8-carbamoyl-3-n-propyl-[3H]-imidazo[5,1-d]-1,2,3,5-tetrazin-4-one;

8-carbamoyl-3-(2-chloroethyl)-[3H]-imidazo[5,1-d]-1,2,3,5-tetrazin-4-one;

3-(2-chloroethyl)-8-methylcarbamoyl-[3H]-imidazo[5,1-d]-1,2,3,5-tetrazin-4-one;

8-carbamoyl-3-(3-chloropropyl)-[3H]-imidazo[5,1-d]-1,2,3,5-tetrazin-4-one;

8-carbamoyl-3-(2,3-dichloropropyl)-[3H]-imidazo[5,1-d]-1,2,3,5-tetrazin-4-one;

3-allyl-8-carbamoyl-[3H]-imidazo[5,1-d]-1,2,3,5-tetrazin-4-one;

3-(2-chloroethyl)-8-dimethylcarbamoyl-[3H]-imidazo[5,1-d]-1,2,3,5-tetrazin-4-one;

3-(2-bromoethyl)-8-carbamoyl-[3H]-imidazo[5,1-d]-1,2,3,5-tetrazin-4-one;

3-benzyl-8-carbamoyl-[3H]-imidazo[5,1-d]-1,2,3,5-tetrazin-4-one;

8-carbamoyl-3-(2-methoxyethyl)-[3H]-imidazo[5,1-d]-1,2,3,5-tetrazin-4-one;

8-carbamoyl-3-cyclohexyl-[3H]-imidazo[5,1-d]-1,2,3,5-tetrazin-4-one;

8-carbamoyl-3-(p-methoxybenzyl)-[3H]-imidazo[5,1-d]-1,2,3,5-tetrazin-4-one; and

8-(N-allylcarbamoyl)-3-(2-chloroethyl)-[3H]-imidazo[5,1-d]-1,2,3,5-tetrazin-4-one.

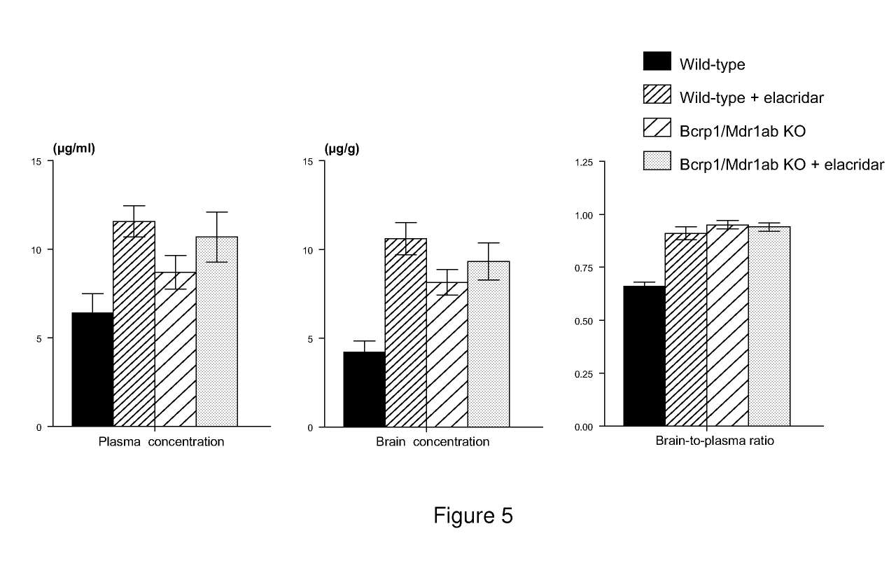

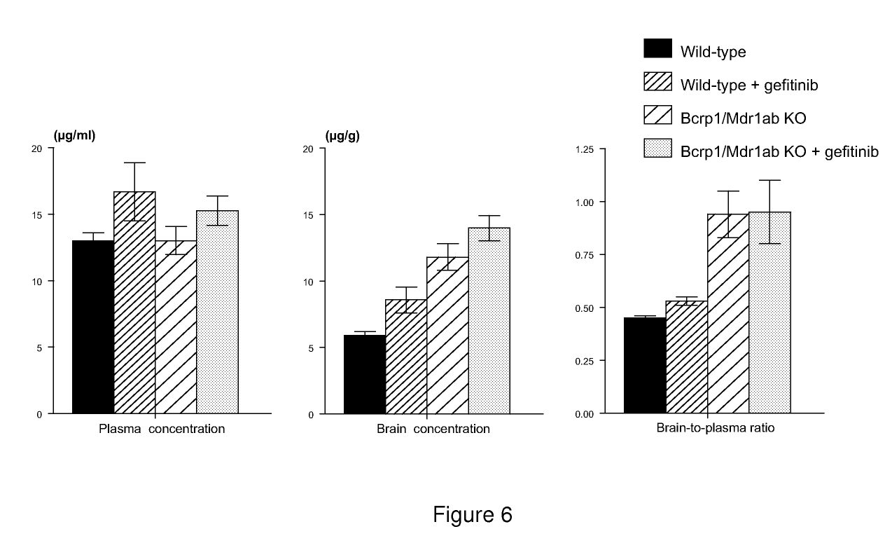

More preferably the imidazotetrazine is 8-carbamoyl-3-methyl-[3H]-imidazo[5,1-d]-1,2,3,5-tetrazin-4-one, i.e. temozolomide having the formula III (FIG. 5).

The imidazotetrazines above also include pharmaceutically acceptable salts and hydrates, or may be crystallised with other solvents used for crystallisation.

Identification of BCRP Inhibitors and P-gp Inhibitors

In a further aspect of the invention there is provided a method of screening for a BCRP inhibitor comprising the steps of:

(a) contacting a cell that expresses BCRP with temozolomide and a candidate inhibitor;

(b) observing or measuring the transport of temozolomide into the cell or through the cell of (a) in the presence of the candidate inhibitor;

(c) comparing said transport of temozolomide in (b) with the transport of temozolomide into the cell or through the cell of (a) in the absence of the candidate inhibitor.

A decrease in transport of temozolomide in the presence of the candidate inhibitor is indicative that the candidate inhibitor is a BCRP inhibitor.

The method may also comprise step (b*), performed prior to step (c): observing or measuring the transport of temozolomide into the cell or through the cell of (a) in the absence of the candidate inhibitor. Alternatively, step (b*) may be performed as a separate experiment. This may have been performed prior to carrying out the method such that the result is a known value. In this case, step (c) involves comparing said transport with a reference value which corresponds to the transport of temozolomide into the cell or through the cell in the absence of the BCRP inhibitor.

Preferably the cell in (a) does not, or substantially does not, transport temozolomide into the cell and/or through the cell when the cell does not express BCRP. In such a cell any increase in transport of temozolomide should be caused by inhibition of BCRP. Thus, the method may also comprise comparing the transport of temozolomide into or through the cell of (a) in the absence of the candidate inhibitor with the transport of temozolomide into or through a cell that does not express BCRP but is otherwise equivalent to the cell of (a).

A cell that does not express BCRP but is otherwise equivalent to the cell of (a) may be readily obtained by a person skilled in the art. For example, the cell of (a) may not naturally express BCRP, e.g. the cell of (a) may have been transduced with BCRP encoding nucleic acid. In this case an otherwise equivalent cell may correspond to the untransduced cell. Alternatively, antisense RNA may be used to down-regulate expression of the BCRP-encoding nucleic acid. The cell may be a Madine Darby Canine Kidney (MDCK) cell.

Preferably the above method of screening for a BCRP inhibitor is an in vitro transport assay, e.g. as described in the Examples below. The in vitro transport assay may be used to determine the IC50 of a BCRP inhibitor and/or a candidate inhibitor, which is the concentration of the inhibitor at which the rate of transport of temozolomide is reduced by 50% compared to the rate of transport of temozolomide in the absence of the inhibitor.

In a further aspect of the invention there is provided a method of screening for a P-gp inhibitor comprising the steps of:

(a′) contacting a cell that expresses P-gp with a P-gp substrate and a candidate inhibitor;

(b′) observing or measuring the transport of the P-gp substrate into the cell or through the cell of (a′) in the presence of the candidate inhibitor;

(c′) comparing said transport of the P-gp substrate in (b′) with the transport of the P-gp substrate into the cell or through the cell of (a′) in the absence of the candidate inhibitor.

The P-gp substrate may be temozolomide, but is preferably digoxine, vinblastine, or paclitaxel in view of our results from using temozolomide as a substrate for P-gp in in vitro transport assays (see below).

A decrease in transport of the P-gp substrate in the presence of the candidate inhibitor is indicative that the candidate inhibitor is a P-gp inhibitor.

The method may also comprise step (b′*), performed prior to step (c′): observing or measuring the transport of the P-gp substrate into the cell or through the cell of (a′) in the absence of the candidate inhibitor. Alternatively, step (b′*) may be performed as a separate experiment. This may have been performed prior to carrying out the method such that the result is a known value. In this case, step (c′) involves comparing said transport with a reference value which corresponds to the transport of the P-gp substrate into the cell or through the cell in the absence of the P-gp inhibitor.

Preferably the cell in (a′) does not, or substantially does not, transport the P-gp substrate into the cell and/or through the cell when the cell does not express P-gp. In such a cell any increase in transport of the P-gp substrate should be caused by inhibition of P-gp. Thus, the method may also comprise comparing the transport of the P-gp substrate into or through the cell of (a′) in the absence of the candidate inhibitor with the transport of the P-gp substrate into or through a cell that does not express P-gp but is otherwise equivalent to the cell of (a′).

A cell that does not express P-gp but is otherwise equivalent to the cell of (a′) may be readily obtained by a person skilled in the art. For example, the cell of (a′) may not naturally express P-gp, e.g. the cell of (a′) may have been transduced with P-gp encoding nucleic acid. In this case an otherwise equivalent cell may correspond to the untransduced cell. Alternatively, antisense RNA may be used to down-regulate expression of the P-gp-encoding nucleic acid. The cell may be a LLC pig-kidney (PK1) cell.

Preferably the above method of screening for a P-gp inhibitor is an in vitro transport assay, e.g. as described in the Examples below in which digoxine, or vinblastine, paclitaxel is used as the P-gp substrate instead of temozolomide. The in vitro transport assay may be used to determine the IC50 of a P-gp inhibitor and/or candidate inhibitor, which is the concentration of the inhibitor at which the rate of transport of the P-gp substrate is reduced by 50% compared to the rate of transport of P-gp substrate in the absence of the inhibitor.

Measuring transport of compounds into a cell is described, for example, in Chen et al. J. Biol. Chem., 2001, 276, 33747-33754.

In a further aspect of the invention there is provided a method of screening for a BCRP and/or a P-gp inhibitor comprising the steps of:

(i) administering a candidate inhibitor and temozolomide to an animal that expresses BCRP and/or P-gp, and preferably expresses BCRP and/or P-gp in the blood brain barrier;

(ii) observing or measuring the amount, e.g. concentration, of temozolomide in the brain of the animal of (i);

(iii) comparing the amount of temozolomide in the brain of the animal in (ii) with the amount of temozolomide in the brain of the animal of (i) administered with the same dose of temozolomide in the absence of the candidate inhibitor.

If the animal of (i) expresses BCRP and P-gp, an increase in the amount of temozolomide in the brain of the animal when the candidate inhibitor is administered to the animal is indicative that the candidate inhibitor is a BCRP inhibitor and/or a P-gp inhibitor. If the animal of (i) expresses BCRP but not P-gp, an increase in the amount of temozolomide in the brain when the candidate inhibitor is administered to the animal is indicative that the candidate inhibitor is a BCRP inhibitor. If the animal of (i) expresses P-gp but not BCRP, an increase in the amount of temozolomide in the brain when the candidate inhibitor is administered to the animal is indicative that the candidate inhibitor is a P-gp inhibitor.

The method may also comprise step (ii*), performed prior to step (iii): observing or measuring the amount of temozolomide in the brain of the animal of (i) in which the same dose of temozolomide is administered to the animal in the absence of the candidate inhibitor. Alternatively, step (ii*) may be performed as a separate experiment. This may have been performed prior to carrying out the method such that the result is a known value. In this case, step (ii*) involves comparing the amount observed or measured in (ii) with a reference value which corresponds to the amount of temozolomide in the brain of the animal of (i) administered with the same dose of temozolomide in the absence of the candidate inhibitor.

Preferably the method additionally comprises sacrificing the animal. Preferably the animal of (i) is an animal to which administration of temozolomide results in a lower amount of temozolomide in the brain compared to an otherwise equivalent animal that is engineered so that it does not express BCRP and/or P-gp. In such an animal any increase in amount of temozolomide in the brain of the animal should be caused by inhibition of BCRP and/or P-gp, respectively. Thus, the method may also comprise comparing the amount of temozolomide in the brain of the animal of (i) with the amount of temozolomide in the brain of an animal that does not express BCRP and/or P-gp but is otherwise equivalent to the animal of (i). BCRP and/or P-gp expression in the animal may be down-regulated using antisense RNA. Alternatively, the BCRP-encoding nucleic acid may be disrupted or removed from the genome of the animal.

Preferably the above method of screening for a BCRP inhibitor and/or a P-gp inhibitor is an in vivo pharmokinetics study as described in the Examples below. Preferably administration of a BCRP or a P-gp inhibitor increases the brain concentration of temozolomide in the animal of (i), e.g. the AUC₀₋₇, by at least 5%, preferably by a least 10%, more preferably by at least 15%, most preferably by at least 20%. Preferably administration of a BCRP inhibitor and a P-gp inhibitor, which may be a single entity, e.g. elacridar, increases the brain concentration of temozolomide in the animal of (i), e.g. the AUC₀₋₇, by at least 20%, preferably by at least 30%, more preferably by at least 40% and most preferably by at least 50%.

Preferably the BCRP inhibitor and/or the P-gp inhibitor or candidate inhibitor is administered to the animal at a non-toxic dosage level. The inhibitor, for example elacridar, may be administered to a mouse in multiple repeated doses at a dose of at least 50 mg/kg, e.g. within the range of 50 mg/kg to 100 mg/kg, e.g. to provide a plasma concentration between 800 ng/ml (at 2 h post dosing) to 400 ng/ml at 24 h. Temozolomide may be administered at 50 mg/kg e.g. within the range of 10 mg/kg to 200 mg/kg, by intravenous injection.

A BCRP inhibitor is preferably identified by performing the method comprising steps (a) to (c) to identify a candidate inhibitor as a BCRP inhibitor in vitro, and then optionally performing the method comprising steps (i) to (iii) to confirm that the candidate inhibitor is a BCRP inhibitor in vivo. Thus, a BCRP inhibitor is preferably an entity or candidate inhibitor whose presence results in a decrease in transport of temozolomide when steps (a) to (c) are performed and whose presence results in an increase in the amount of temozolomide in the brain of an animal when steps (i) to (iii) are performed, e.g. by at least 10%, using a non-toxic dosage level.

Likewise, a P-gp inhibitor is preferably identified by performing the method comprising steps (a′) to (c′) to identify a candidate inhibitor as a P-gp inhibitor in vitro, and then optionally performing the method comprising steps (i) to (iii) to confirm that the candidate inhibitor is a P-gp inhibitor in vivo. Thus, a P-gp inhibitor is preferably an entity or candidate inhibitor whose presence results in a decrease in transport of temozolomide when steps (a′) to (c′) are performed and whose presence results in an increase in the amount of temozolomide in the brain of an animal when steps (i) to (iii) are performed, e.g. by at least 10% using a non-toxic dosage level.

A frequently used way to identify Pgp and/or BCRP inhibitors by people skilled in the art is based on cytotoxicity assays with cells that express one or both of these drug transporters. Cells are exposed to a potent cytotoxic substrate (e.g. paclitaxel or topotecan) in the absence and presence of a putative inhibitor. Ideally the cell is also available as a ‘parental’ cell line that does not express the transporter. Thus for example, cell line A is transduced with MDR1 to yield cell line A-MDR1 expressing human P-gp. When exposed to a cytotoxic P-gp-substrate, A-MDR1 will be less sensitive than A. When they are exposed to the same drug in combination with a P-gp inhibitor the sensitivity of A will not change, but A-MDR1 will become more sensitive. Ideally, at full inhibition the A-MDR1 cell line will become as sensitive as A. Cytotxicity can be measured by any proliferative test (e.g. MTT) or clonogenic assay. The concentration of the cytotoxic substrate required to reduce proliferation/clonogenicity by 50% is the IC50 of the cytotoxic substrate. The IC50 value of A-MDR1 vs A is called the resistance factor (RF). The concentration of the inhibitor required to reduce this resistance factor by 90% can be used as a measure for the potency of the inhibitor.

Methods according to the present invention may be performed in vitro or in vivo. The term “in vitro” is intended to encompass experiments with cells in culture whereas the term “in vivo” is intended to encompass experiments with intact multi-cellular organisms. Where the method is performed in vitro it may comprise a high throughput screening assay. Candidate inhibitors used in a method described herein may be obtained from a synthetic combinatorial peptide library, or may be synthetic peptides or peptide mimetic molecules. Candidate inhibitors may also be antibodies from a phage display library or a polyclonal sample of antibodies, or may be a pool of nucleic acids incorporating a random nucleotide sequence. Other candidate inhibitors may comprise defined chemical entities, oligonucleotides or nucleic acid ligands (aptamers).

Combination Therapy

A BCRP inhibitor and/or a P-gp inhibitor, and an imidazotetrazine may be administered as a single composition or as different compositions. In other words, the BCRP inhibitor and/or the P-gp inhibitor and the imidazotetrazine may each be present in a different composition. When administration involves administering a BCRP inhibitor, a P-gp inhibitor, and an imidazotetrazine, two of these may be present in a single composition and the third in a different composition, or all three may be present in same composition, or all three may be present in separate compositions. Administration of a BCRP inhibitor and/or a P-gp inhibitor and an imidazotetrazine may be simultaneous or separate (chronologically staggered), e.g. at different time points with equal or different time intervals. Preferably the time intervals are chosen such that the therapeutic effect on the cancer of the combined use of the BCRP inhibitor and/or the P-gp inhibitor and the imidazotetrazine is greater than that which would be obtained by administration of the imidazotetrazine alone.

Cancer

The cancer to be treated may be any unwanted cell proliferation (or any disease manifesting itself by unwanted cell proliferation), neoplasm or tumour or increased risk of or predisposition to the unwanted cell proliferation, neoplasm or tumour. The cancer may be a benign or malignant cancer and may be primary or metastatic. A neoplasm or tumour may be any abnormal growth or proliferation of cells and may be located in any tissue. Examples of tissues include the colon, pancreas, lung, breast, uterus, stomach, kidney, testis, central nervous system, e.g. the brain, peripheral nervous system, skin (melanoma), blood or lymph. The cancer may be a tumour that is sensitive to temozolomide, i.e. that responds to treatment with temozolomide.

For example, the cancer may be a tumour (e.g. a primary tumour) of the central or peripheral nervous system, e.g. glioma, medulloblastoma, meningioma, neurofibroma, ependymoma, Schwannoma, neurofibrosarcoma, astrocytoma or oligodendroglioma. The cancer may also be a tumour (e.g. a primary tumour) of a tissue that shows sensitivity to temozolomide, i.e. temozolomide inhibits proliferation of the tumour cells, and that is known to metastasise to the brain, e.g. melanoma. The cancer preferably occurs in the central nervous system, e.g. the brain. Preferably the cancer is glioma, high-grade glioma, glioblastoma, e.g. glioblastoma multiforme (GBM).

GBM may be primary or secondary GBM. Generally, secondary GBM starts as a grade II glioma (i.e. WHO grade II), which after many years (e.g. up to 10-15) will evolve into a high-grade glioma and glioblastoma multiforme (GBM). In primary GBM patients will usually already present high-grade glioma at the time of diagnosis without evidence that they have a previous history of low-grade glioma. Glioblastoma and its variants correspond to WHO grade IV. (For a discussion and the WHO definition of Glioblastoma see World Health Organisation Classification of Tumours, Pathology & Genetics, Tumours of the Nervous System, Edited by Paul Kleihues & Webster K. Cavenee, International Agency for Research on Cancer (IARC) Press, Lyon, 2000.)

Alternatively, the cancer may be a metastatic cancer, e.g. one occurring in the brain but originating from a primary cancer located elsewhere in the patient's body, and preferably outside the CNS, e.g. metastatic melanoma.

Chemotherapeutic agents are agents, e.g. pharmaceuticals, that may be used to treat cancer, e.g. the agents have antineoplastic activity such that they are able to inhibit the proliferation of tumour cells.

Patients

The patient to be treated may be any animal or human. The patient may be a non-human mammal, but is more preferably a human patient. The patient may be male or female.

Aptamers

Aptamers, or nucleic acid ligands, are nucleic acid molecules characterised by the ability to bind to a target molecule with high specificity and high affinity. Aptamers to a given target may be identified by the method of Systematic Evolution of Ligands by Exponential enrichment (SELEX™). Aptamers and SELEX are described in WO91/19813.

Aptamers may be DNA or RNA molecules and may be single stranded or double stranded. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers can be thought of as the nucleic acid equivalent of monoclonal antibodies and often have K_{d}'s in the nM or pM range. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule.

RNA Interference (RNAi)

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAS” (siRNAs) or “microRNAs” (miRNAs) depending on their origin. Both types of sequence may be used to down-regulate gene expression by binding to complimentary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene down-regulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of BCRP or P-gp. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of BCRP or P-gp.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

In one embodiment, the vector may comprise a nucleic acid sequence according to the invention in both the sense and antisense orientation, such that when expressed as RNA the sense and antisense sections will associate to form a double stranded RNA. In another embodiment, the sense and antisense sequences are provided on different vectors.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through-O-or-S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus modified nucleotides may also include 2′substituted sugars such as 2′-O-methyl-; 2-O-alkyl ; 2-O-allyl ; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

Formulating Pharmaceutically Useful Compositions and Medicaments

In accordance with the present invention methods are also provided for the production of pharmaceutically useful compositions based on a BCRP inhibitor and/or a P-gp inhibitor. In addition to the steps of the methods described herein, such methods of production may further comprise one or more steps selected from:
(a) identifying and/or characterising the structure of a BCRP inhibitor and/or P-gp inhibitor;
(b) obtaining the BCRP inhibitor and/or P-gp inhibitor;
(c) mixing the BCRP inhibitor and/or P-gp inhibitor with an imidazotetrazine and/or a pharmaceutically acceptable carrier, adjuvant or diluent.

For example, a further aspect of the present invention relates to a method of formulating or producing a pharmaceutical composition for use in the treatment of cancer, the method comprising identifying a BCRP inhibitor or a P-gp inhibitor in accordance with the methods described herein, and further comprising one or more of the steps of:
(i) identifying the BCRP inhibitor and/or P-gp inhibitor; and/or
(ii) formulating a pharmaceutical composition by mixing the BCRP inhibitor and/or P-gp inhibitor, or a prodrug thereof, with an imidazotetrazine and/or a pharmaceutically acceptable carrier, adjuvant or diluent.

The active ingredients may be present in free form or in the form of a pharmaceutically acceptably salt.

Certain pharmaceutical compositions formulated by such methods may comprise a prodrug of the BCRP inhibitor and/or the P-gp inhibitor wherein the prodrug is convertible in the human or animal body to the desired active agent. In other cases the active agent may be present in the pharmaceutical composition so produced and may be present in the form of a physiologically acceptable salt.

Administration

The BCRP inhibitors and/or P-gp inhibitors and/or imidazotetrazines of the invention may be formulated for topical, parenteral, systemic, intravenous, intra-arterial, intramuscular, intrathecal, intraocular, intratumoural, subcutaneous, oral or transdermal routes of administration which may include injection. Injectable formulations may comprise the selected compound in a sterile or isotonic medium.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Treatment regimes for temozolomide are known in the art. For example, in one treatment regimen, temozolomide is given for 5 days followed by a 23-day rest period. In another regimen temozolomide is given daily for 42 days. Alternatively, patients may start with temozolomide (e.g. 75 mg/m²/day for 42 days) concomitantly with radiotherapy, subsequently followed by temozolomide monotherapy (e.g. 150 to 200 mg/m²/day for 5 days with a 23 day rest period).

Preferably a BCRP inhibitor and/or a P-gp inhibitor is administered together with the temozolomide, e.g. on the same day. Preferably the BCRP inhibitor and/or the P-gp inhibitor or candidate inhibitor is administered at a non-toxic dosage level. For example, the BCRP inhibitor and/or P-gp inhibitor, e.g. elacridar, may be administered to a human at a dose of 1000 mg per os (oral), e.g. to result in plasma levels of about 400-500 ng/ml at 8 h post drug administration to 100 ng/ml at 24 h. For example, elacridar may be administered at a dose of 1000 mg per os (oral) 8 hours prior to administration with temozolomide. We understand that elacridar is safe in humans, which may allow further dose escalation (Kruytzer et al J. Clin Oncol. 20: 2943-2950, 2002).

Alternatively, targeting therapies may be used to deliver the active agent more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Specific details of the best mode contemplated by the inventors for carrying out the invention are set forth below, by way of example. It will be apparent to one skilled in the art that the present invention may be practiced without limitation to these specific details.

### EXAMPLES

Materials and Methods

Reagents

Temozolomide (Temodal® 20 mg hard capsules) originated from Schering Plough BV (Utrecht The Netherlands). Elacridar (GF120918) was a generous gift from GlaxoSmithKline Wellcome, Inc. (Research Triangle Park, N.C., USA). Erlotinib was a generous gift of OSI Pharmaceuticals, Inc., Melville, N.Y., USA). Zosuquidar was a generous gift of Eli Lilly and Company (Indianapolis, Ind., USA). Gefitinib was purchased from Sequoia Research Products Ltd (Pangbourne, UK). Bovine Serum Albumin (BSA), fraction V, was purchased from Roche Diagnostics GmbH (Mannheim, Germany). All other chemicals were purchased from E. Merck (Darmstadt, Germany) and were used as supplied. Water was purified by the Milli-Q Plus® system (Millipore, Milford, USA).

Preparation of Drug Solutions

The contents of a temozolomide capsule containing 20 mg of active substance was dissolved in 0.4 ml ethanol and 3.6 ml saline to yield a solution of 5.0 mg/ml. Elacridar (GF120918) was prepared freshly the day before each experiment and suspended at 5 mg/ml in a mixture of hydroxypropyl methylcellulose (0.5 g/l)/1% polysorbate 80 (v/v). The suspension was mixed for 2 minutes using a Polytron PT1200 homogenizer (Kinematica AG, Littau, Switzerland). Additionally, the suspension was kept protected from light and stirred continuously before and during administration. Gefitinib was suspended in 0.5% (v/v) Tween 20 and 0.25% (w/v) carboxymethylcellulose in water at a concentration of 10 mg/ml.

Analytical Methods

Based on previous work by Kim et al (11) we have developed a high-performance liquid chromatographic assay for the determination for quantification of temozolomide in medium used for transwell experiments and in mouse plasma and brain tissue homogenates for in vivo pharmacokinetic studies. Separation/quantification was achieved using a Symmetry® C₁₈ column (150×2.0 mm; ID) together with a mobile phase of 7.5% of methanol in 0.5% acetic acid in water, delivered at a flow rate of 0.2 ml/min and UV detection at 330 nm ((PDA996 photodiode array detector; Waters, Milford, Mass., USA or SF 757 detector; Kratos, Ramsey, N.J., USA). Medium from transwell experiments was 10-fold diluted with 0.2% acetic acid in water and 50 μl was injected directly into the HPLC system. Temozolomide was extracted from the acidified plasma and brain tissue homogenate samples (200 μl) with 1.0 ml ethyl acetate. The dried extracts were subsequently dissolved in 100 μl of 5% methanol in 0.2% acetic acid in water and 50 μl was injected into the HPLC system. External calibration was performed since no internal standard was available. The lower and upper limit of quantitation was 0.020 and 10.0 μg/ml, respectively. Samples above the upper limit of quantification were first diluted with acidified blank human plasma. All samples from in vivo studies were analyzed in duplicate in 2 independent analytical series and repeated once more when the results differed by more than 10%.

In Vitro Transport Experiments

The parental LLC pig-kidney (PK1) cell line and sublines transduced with murine Mdr1a or human MDR1 (12) and the parental Madine Darby Canine Kidney (MDCK) and murine Bcrp1 or human BCRP transduced sublines (13) were seeded on Transwell microporous polycarbonate membrane filters (3.0 μm pore size, 24 mm diameter; Costar Corning, N.Y., USA) at a density of 1×10⁶ cells per well in 2 ml of MEM medium (Invitrogen Corporation, Carlsbad, Calif.) containing 10% v/v fetal calf serum. Cells were incubated at 37° C. in 5% CO₂ for three days with one medium replacement after the first day. Two hours before the start of the experiment the medium in both compartments was replaced with 2 ml of OptiMEM medium (InVitrogen Corp.). At the start of the experiment the medium in the apical or basolateral compartment was replaced with 2 ml of freshly prepared OptiMEM medium containing 40 μg/ml of temozolomide. The P-gp and/or BCRP inhibitors elacridar (5 μM), gefitinib (5, 10 μM), erlotinib (5, 10 and 20 μM) or novobiocin (10 and 50 μ) were added to both the apical and basolateral compartment prior to temozolomide. Zosuquidar (LY335979, 5 μM) was always added to the medium when doing experiments with MDCK cell lines to inhibit endogenous canine P-gp. Samples of 50 μl were collected every 30 min, for up to 2 h after start of the experiment. Because temozolomide is instable in medium at 37° C., we replaced one ml of the donor compartment by a freshly prepared aliquot every 30 min. [³H]-inulin (approximately 7 kBq per well) was added to the same compartment as temozolomide to check the integrity of the cell layer. Wells showing a leakage of more than 1.5% per hour were excluded.

Animals

Animals used in the pharmacokinetics studies were male wild-type, Bcrp1 knockout, Mdr1ab knockout and Bcrp1/Mdr1ab knockout mice of a FVB genetic background within the age range of 8 to 15 weeks. Animals used for efficacy studies were athymic (nude) mice of FVB background of wild-type or Bcrp1/Mdr1ab knockout genotype.Animals were housed and handled according to institutional guidelines complying with Dutch law. The mice were kept in a temperature-controlled environment with a 12-hour light-12-hour dark cycle and were given a standard diet (AM-II; Hope Farms B.V., Woerden, The Netherlands) and acidified water was provided ad libitum. All experiment involving animals were approved by the local animal ethics committee.

In Vivo Pharmacokinetics Studies

The brain penetration study comprised cohorts of animals receiving temozolomide (50 mg/kg) by intravenous injection in the tail vein. Each cohort consisted of at least 40 animals in which at least 6 animals were used per time point (t=15 min, 1, 2, 4 and 7 hours post temozolomide administration). Separate cohorts of wild-type and P-gp/Bcrp1 knockout mice received temozolomide (50 mg/kg) as single agent versus 2 h after elacridar (100 mg/kg) administered orally by gavage into the stomach or a single agent versus 1 h after oral gefitinib (100 mg/kg). The mice were anesthetized with metoxyflurane and blood samples were obtained by cardiac puncture and collected on ice in tubes containing potassium EDTA as anticoagulant. The tubes were placed in melting ice, centrifuged within 60 min (10 min, 5000 g, 4° C.) to separate the plasma fraction, which was transferred into clean vials, mixed with 1 M hydrochloric acid (10+1; v/v) and stored at −20° C. until analysis. Immediately after cardiac puncture the mice were killed by cervical dislocation and the brains were dissected and placed on ice. Within 60 min they were weighed and homogenized in 3 ml of ice-cold 1% of BSA in 0.05 M phosphate buffer adjusted to pH 2 and stored at −20° C. until further analysis.

We also established the drug exposure in wild-type (n=18) and Bcrp1/Mdr1ab knockout mice (n=19) receiving an oral dose of 100 mg/kg by gavage. Blood was sampled from the tail at 15, 30 min and 1, 4 and 7 h post drug administration to obtain a full curve from each animal.

Tumor Xenograft Studies

Intracranial Mel57-luc (luciferase transfected subline) tumor xenografts in wild-type nude mice (WT) and mdr1ab knockout nude mice (MDR) were established according to the protocol below (see Kemper et al, Eur J Cancer 42:3294-3303, 2006).

The Mel57 cell line (University Medical Centre, Nijmegen, The Netherlands) is a human melanoma cell line and was cultured in MEM (Life Technologies Inc., Breda, The Netherlands) supplemented with 10% foetal calf serum, non-essential amino acids, streptomycin, penicillin and sodium pyruvate (all Life Technologies), and incubated at 37° C. and 5% CO₂ in humidified air. The cell line was further transfected by standard protocols with lipofectamine using a pBS vector containing a β-actin driven luciferase gene coupled via an internal ribosome expression site to GFP. After expansion, GFP bright cells were sorted to single cells by FACS, expanded and sublines tested for luciferase expression by an in vitro bioluminescence assay (Promega, Benelux BV, Leiden, The Netherlands). Three to five sublines showing the highest luminescence activity were tested in vivo for tumour take and intracranial growth behaviour. To retrieve tumour cells for injection, the cell culture was trypsinised (trypsin 0.05%; EDTA 0.02%, Life Technologies), washed two times with Hanks Balanced Saline Solution (HBSS, Life Technologies Inc.), counted and resuspended in an appropriate volume of HBSS. The single cell suspension was kept on ice until injection.

Mice were anaesthetised by intraperitoneal injection of 6 ml/kg of a 1:1:2 (v/v/v) mixture of Hypnorm (fentanyl 0.2 mg/ml and fluanisone 10 mg/ml; Jansen Cilag, Tilburg, The Netherlands), Dormicum (midazolam 5 mg/ml; Roche Nederland, Mijdrecht, The Netherlands) and water for injection (Braun, Emer-Compascuum, The Netherlands). Mice were placed in a stereotact (ASI instruments, Warren, Mich., US). After preparation of the skull a small hole of 1 mm in diameter was drilled at 2 mm lateral and lmm anterior to the bregma, because this was shown to be a reliable place for tumour engraftment (Lai S. et al, J. Neurosurg, 2000, 92:326-33). The 30-gauge needle was attached to a 50 μl syringe (Hamilton Bonaduz AG, Bonaduz, Switzerland) and fitted in an infusion pump (Baby Bee Syringe Drive (BAS Instruments Ltd. Warwickshire, UK)). The needle was inserted 3 mm below the skull surface and 2 μl of a cell suspension containing 1×10⁵ Mel57 cells was injected, at a rate of 0.4 μl/min.

Mel57-luc cells (100,000 cells/2 μl) were injected stereotactically in the brain as described in detail in Kemper et al¹⁴. Seven days later, bioluminescence imaging using an IVIS200 camera (Caliper Life Science, Alameda, Calif., USA) was performed to establish tumor load in each animal at the start of therapy. The animals were stratified according to genotype and tumor load to receive oral temozolomide 100 mg/kg/day×5 by gavage or no treatment. Bioluminescence imaging was repeated at subsequent days to establish the efficacy of the therapy. The amount of bioluminescence in each animal was calculated relative to the first measurement when therapy was initiated (arbitrarily set at 100%) and was log converted prior to data analysis. Tumor volume was measured by bioluminescence activity (see Kemper et al, Eur J Cancer 42:3294-3303, 2006).

Data Analysis

Plasma and brain AUC₀₋₇ₕ values and standard errors were calculated by the linear trapezoidal rule using standard equations (14). The plasma half-life of temozolomide was calculated by linear regression analysis after log transformation of the concentration data using Microsoft Excel 2003. The brain-to-plasma ratio was calculated for each individual animal. The two-sided unpaired Student's t-test was used for statistical analysis of AUC values. Bonferroni's test for multiple comparisons was calculated using SPSS (v12.0.1; SPSS Inc, Chicago, Ill., USA) for brain and plasma levels and brain-to-plasma ratios determined at 2 h post temozolomide administration with the inhibitors and for analyzing the results of the in vivo efficacy study.

Results

In Vitro Transport Assays

Because the stability of temozolomide in culture medium at 37° C. is limited (half-life about 30 min), transwell experiments were carried out slightly differently than usual for this kind of experiment. First, we took samples every 30 min for up to 2 h instead of every hour for up to 4 h. Secondly, to maintain adequate temozolomide levels at the donor side throughout the experiment we replaced 1 ml of the 2 ml of medium at the donor side of the transwell at each sampling time by 1 ml of a freshly prepared temozolomide solution in medium.

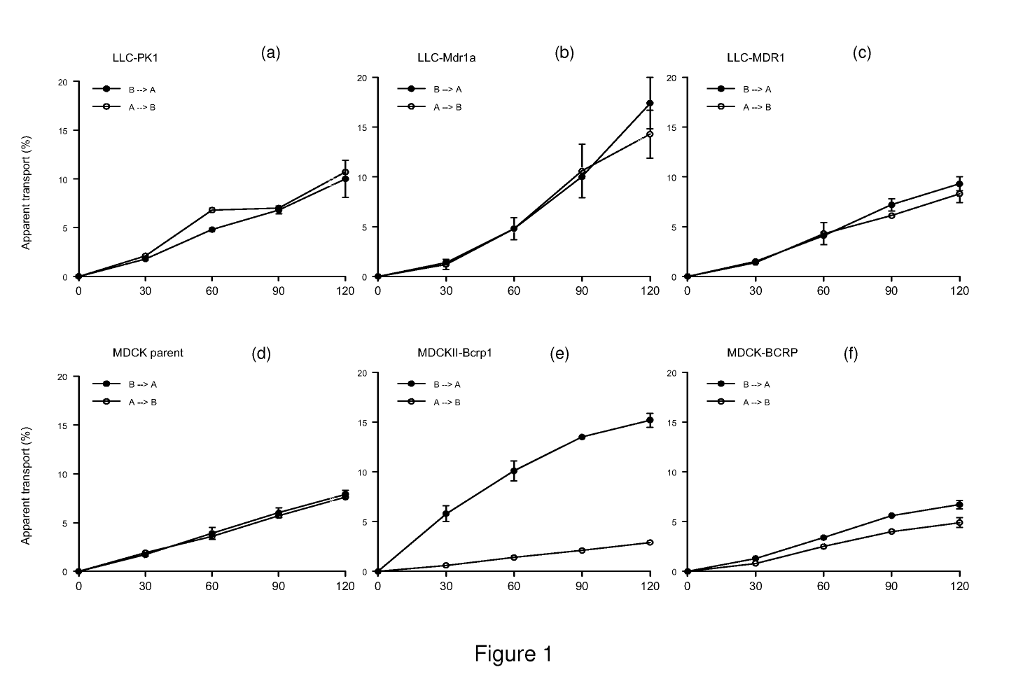

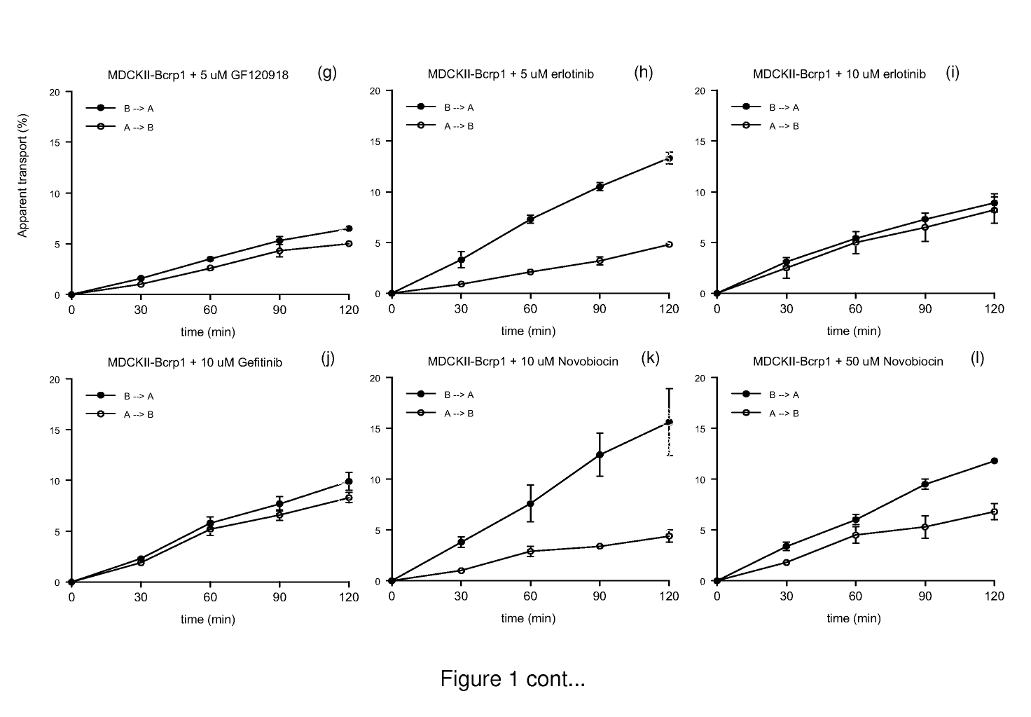

We have investigated transport of temozolomide (40 μg/ml) using polarized monolayers of porcine kidney (LLC-PK1) cells and its murine (LLC-Mdr1a) and human (LLC-MDR1) P-gp transduced subclones showing very limited if any evidence for transport by murine or human P-gp (see FIG. 1). Similar experiments were conducted in the parental canine MDCK cells and its transduced murine Bcrp1 or human BCRP sublines. Whereas murine MDCK-Bcrp1 cells showed clear vectorial transport, more moderate transport was seen in the human BCRP subline. Although this would suggest that temozolomide is a weaker substrate of human BCRP than of mouse Bcrp1 this is not certain, since a much less efficient translocation by the human BCRP transduced subline is seen with all drugs we have tested so far (results not shown) and probably reflects a lower expression level and/or not completely apical location of BCRP in this subline. Bcrp1 mediated transport was (almost) completely abrogated when elacridar (GF120918; 5 μM), gefitinib (10 μM) or erlotinib (10 μM) was present in the medium. A concentration of 50 μM of novobiocin was not sufficient. Together with these experiments we ran control experiments using well-established P-gp and BCRP substrates that were available to us as radioactively labeled compounds (¹⁴C-trabectedin and ¹⁴C-Indisulan, or ¹⁴C-topotecan). These experiments showed that 20 μM of gefitinib was sufficient to inhibit P-gp mediated transport (results not shown). Moreover, the integrity of all monolayers was checked using ³H-inuline as marker compound. Wells showing more than 1.5% translocation of inuline per 30 min were considered to be leaky and excluded from the analysis. These in vitro results clearly show that whereas temozolomide is a good substrate of Bcrp1 it is much less efficiently transported by P-gp.

In Vivo Pharmacokinetics

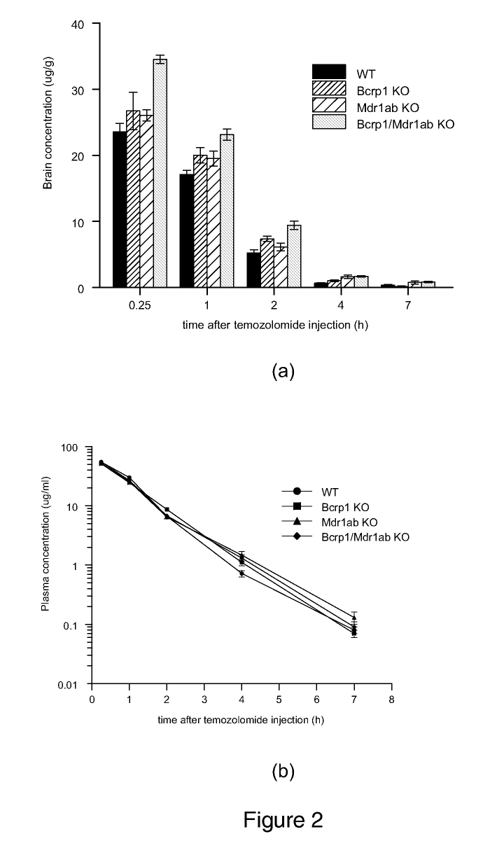

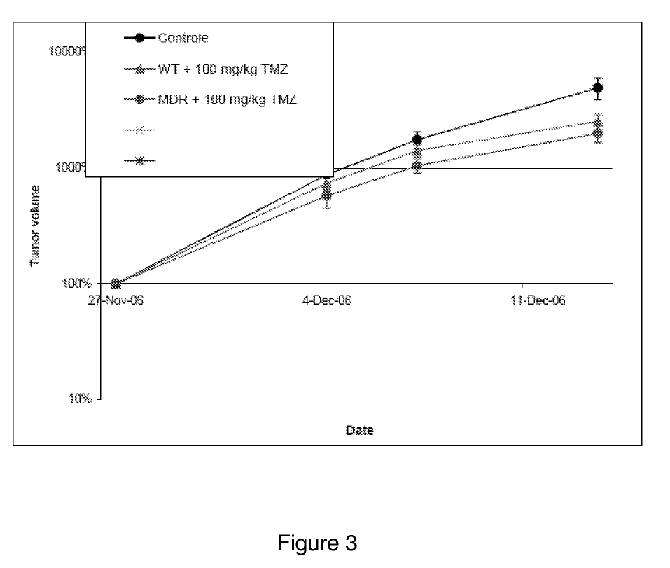

To investigate the impact of Bcrp1 and P-gp on the disposition of temozolomide in vivo we have performed experiments in wild-type, Bcrp1 knockout, Mdr1ab knockout and compound Bcrp1/Mdr1ab knockout mice. All animals received temozolomide by i.v. injection in the tail vein in order to minimize the inter-animal variability that might be higher after oral dosing, although we acknowledge that the oral route is the standard route for clinical use of this drug. Interestingly, the brain accumulation of temozolomide was significantly (P<0.01) higher in both of the single knockout mice strains compared to the wild-type control group (table 1). Although the in vitro results suggested that P-gp does not transport temozolomide the brain of Mdr1ab knockout mice accumulated about 20% more temozolomide. The same enhancement was seen in Bcrp1 knockout mice, whereas compound Bcrp1/Mdr1ab knockout mice accumulated 50% more (P<0.001) drug in the brain (FIG. 2). Although a 50% increase is modest compared to results previously observed with other substrate drugs, we expect this to be highly relevant for treatment of brain cancer patients. The absence of drug transporters did not affect the plasma clearance of this drug (Table 1) nullifying the chance that the higher brain levels are due to higher plasma levels. In fact the decline of temozolomide from plasma follows first-order elimination kinetics with a half-life of 0.7 h, in line with the fact that temozolomide is unstable at physiological pH being non-enzymatically degraded into its metabolite 3-methyl-(triazen-1-yl)imidazole-4-carboximide (15).

To investigate the possibility of enhancing the brain accumulation of temozolomide by inhibition of P-gp and BCRP we have used the dual P-gp/BCRP inhibitor elacridar. Elacridar, given to wild-type mice at a single oral dose of 100 mg/kg resulted in temozolomide brain-to-plasma ratios that were significantly (P=0.001) higher than wild-type controls and similar to those achieved in compound Bcrp1/Mdr1ab knockout mice (FIG. 5). Moreover, no further significant enhancement in the brain penetration of temozolomide was seen when elacridar was given to Bcrp1/Mdr1ab knockout mice, demonstrating that the interaction by elacridar was selective for Bcrp and P-gp.

Gefitinib and erlotinib are epidermal growth factor receptor (EGFR) tyrosine kinase inhibitors that are currently also being tested in patients suffering from GBM also in combination with temozolomide ¹⁸. Based on reports suggesting that these compounds may be relatively potent BCRP and P-gp inhibitors ¹⁹, we have investigated the effect of concomitant gefitinib on the brain penetration of temozolomide. However, gefitinib given at 100 mg/kg, did not significantly enhance the brain-to-plasma ratio of temozolomide.

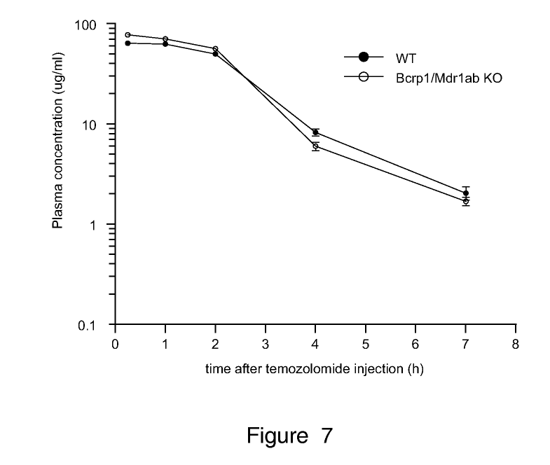

Since both P-gp and BCRP may limit the oral bioavailability of substrate drugs, we have investigated the drug exposure of oral temozolomide in wild-type versus Bcrp1/Mdr1ab knockout mice using the dose that would be used in the subsequent in vivo efficacy study against intracranial xenografts (FIG. 7). Again, the plasma AUC was not significantly higher in the Bcrp1/Mdr1ab knockout mice (Table 2).

Tumor Xenografts

To confirm the relevance of the higher brain penetration for treatment of intracranial tumors we performed an in vivo efficacy study using Mel57 melanoma cell line. By using gadolinium-DTPA magnetic resonance imaging we have previously shown that the BBB within these brain lesions is relatively intact²³.

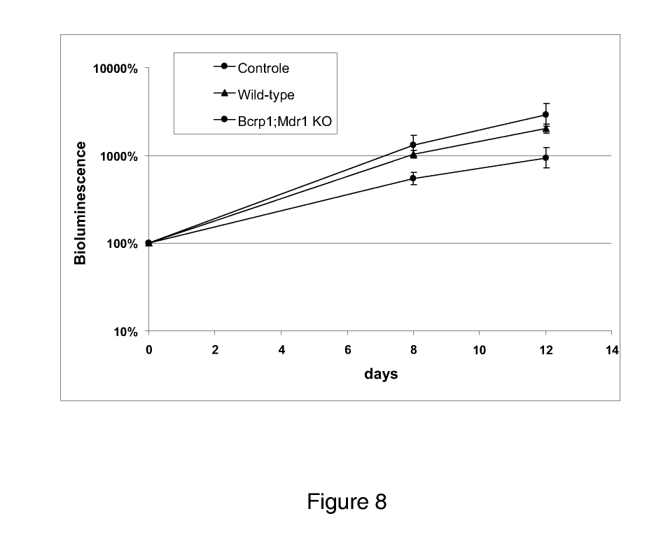

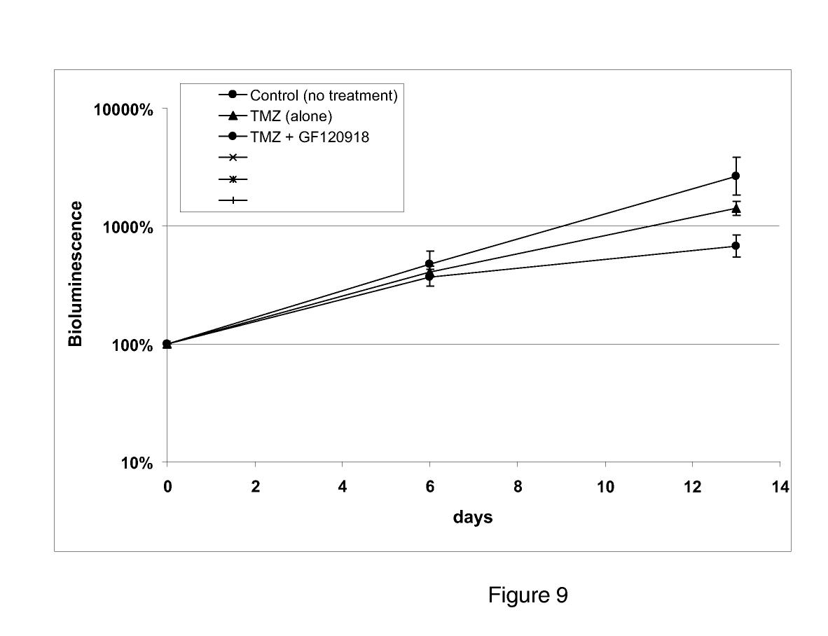

We investigated the efficacy of temozolomide against intracranial Mel57-luc (luciferase transfected subline) tumor xenografts in wild-type nude mice (WT) and mdrlab knockout nude mice (MDR). Mel57 is a human melanoma cell line that when grafted in the brain of mice will have a non-leaky blood-brain barrier (see Kemper et al, Eur J Cancer 42:3294-3303, 2006). The results show that mice that are deficient in P-gp respond better to temozolomide chemotherapy than the proficient P-gp mice. As these mice still express Bcrp1 in the BBB, we expected that the response in Bcrp1/Mdr1ab knockout mice would further improve. This was confirmed in a subsequent experiment using Bcrp1/Mdr1ab knockout nude mice where the difference in efficacy of temozolomide therapy between wild-type and Bcrp1/Mdr1a/b was more pronounced (FIG. 8).

Discussion

This study shows that the absence of both P-gp and Bcrp1 enhances the brain penetration of temozolomide by 50 percent without altering the body clearance of this drug. A similar effect was seen in wild-type mice that received the dual P-gp and BCRP inhibitor elacridar. The increased brain penetration translated into a significantly better antitumor response in an experimental intracranial tumor model. Most likely, this 50% gain in brain penetration of this drug that already elicits meaningful clinical responses against primary brain cancer may provide another step forward in the treatment of this devastating disease. Consequently, our results provide a solid basis for clinical testing of combinations of P-gp/BCRP inhibitors (e.g. elacridar) and temozolomide in patients suffering from malignant glioma.

The finding that temozolomide is a substrate of P-gp was not very clear from the in vitro results. Usually, the difference between apical-to-basal versus basal-to-apical transport is much more profound. Most likely, temozolomide is a weaker substrate of P-gp than paclitaxel or other typical substrate drugs (12;14). The affinity of temozolomide for murine Bcrp1 was more convincingly shown by the in vitro results. The lack of finding at least some transport by P-gp may also be related to the instability of temozolomide in the transport medium that has a pH of 7.4. Because of the instability of temozolomide in the transport medium it is not possible to calculate the permeability in moles per cm², but taking into account this instability, the finding that at least 5% of the dose is recovered in the acceptor compartment after 30 min suggests that temozolomide readily permeates membranes. The lower apparent transport by human BCRP is probably an underestimation of the real capacity of human BCRP relative to the murine isoform. Most if not all compounds that have been tested by us and others in our institute were much less efficiently translocated by this cell line, indicating that the location/expression of BCRP in this subline is different from that of Bcrp1.

Importantly, however, despite this relatively weak affinity, especially for P-gp, the presence of these drug transporters in the BBB still significantly reduces the brain penetration of temozolomide. This result stresses the efficiency by which these transporters operate at the BBB. Drug transporters appear to be much more efficient in restricting the entry of substrate drugs into the brain than preventing uptake from the gut. For example, the substrate drug imatinib has an excellent oral bioavailability (>90%, (18)), but a poor brain penetration that is significantly enhanced in Bcrp1/Mdr1 knockout mice (24).

Previous studies have demonstrated that the brain-to-plasma ratio of paclitaxel in Mdr1ab knockout mice was more than 5-fold that in wild-type controls ¹⁴. In that perspective, the 1.5-fold (e.g. 50%) enhancement of the brain penetration of temozolomide may seem very modest at first glimpse. However, the fact that temozolomide is an active agent (one of the very few) against GBM indicates that the brain penetration is already sufficient and probably higher than that of many other drugs. Most likely, temozolomide would have been an inactive drug against GBM, had its affinity for P-gp and/or BCRP been more similar to that of paclitaxel and P-gp.

The improved BBB penetration of temozolomide translated into a better tumor response using an intracranial tumor model. Although the Mel57 is not a GBM derived cell line such as for example the frequently used U87 cell line, we have chosen for the Mel57 line because brain tumors formed by these cells have a functional BBB²³. Whereas GBM is characterized by regions where VEGF driven microvascular proliferation results in a disrupted BBB, it is also a very invasive tumor and especially this invasive component of the tumor renders this disease incurable by current treatment modalities. In order to target these invasive tumor cells, agents should be capable to penetrate the BBB. Consequently, it is important to test the antitumor efficacy of agents using models that do not have a very leaky BBB as this will result in an overestimation of the potency of the agent under study. Tumors with leaky vessels, such as U87 are very sensitive to temozolomide. In fact, one single dose of 25 mg/kg of temozolomide was already sufficient to cause a significant antitumor response against this tumor (results not shown).

Although the clearance of temozolomide was unaffected, the combined use of temozolomide with P-gp and BCRP inhibitors may have an impact on the toxicity profile. Currently, two regimens of temozolomide are being used for treatment of brain tumors. In recurrent GBM, the intermittent scheme, temozolomide (150-200 mg/m²/day) is given for 5 days followed by a 23-day rest period, whereas the more continuous schedule of temozolomide (75 mg/m²/day) for 42 days with concomitant radiotherapy is applied for newly diagnosed GBM patients. Besides, reports where temozolomide (150 mg/m²) was given every 2 weeks for 7 days followed by 7 days rest period suggested that further dose-intensification may improve the efficacy of this drug²⁵. All schedules have a good safety profile, with bone marrow toxicity as main dose-limiting toxicity, and it is not clear whether any of these schedules is really superior over the other (20). Given that P-gp and BCRP are expressed in bone marrow stem cells (21;22), their long-term inhibition in combination with the administration of a cytotoxic drug may enhance the myelotoxic effects. We expect that this effect will be less when using the intermittent temozolomide 5-days schedule. Besides the expression of drug transporters, stem cells are also relatively insensitive to chemotherapeutic drugs because of their low proliferation. Upon peripheral neutropenia feedback signaling may drive stem cell proliferation to recruit more progenitor cells, and these cycling stem cells may be more vulnerable to cytotoxic drugs. However, as the white blood cell nadir occurs days after the last dosing at a time that temozolomide and the inhibitor are already cleared from the body the additional toxic effects on the stem cell population may be minimal. This, however, may be different when the drug combination will be given for a period of 42 days.

In conclusion, we have demonstrated that the brain penetration of temozolomide is increased by removing BCRP and/or P-gp function and therefore may be increased by concomitant inhibition of P-gp and Bcrp1. We expect that this combination will further enhance the efficacy of temozolomide against GBM, which should obviously be explored in typical Phase I/II design.

TABLE 1
Pharmacokinetic parameters of temozolomide after i.v.
administration of 50 mg/kg.

| | AUC_{0-7 h, brain} | AUC_{0-7 h plasma} | |
|---|---|---|---|
| Genotype | μg/g · h | μg/ml · h | T½ₚₗₐₛₘₐ h |
| Wild-type | 37.73 ± 1.11 | 65.40 ± 2.00 | 0.689 ± 0.019 |
| Bcrp1 | 44.78 ± 1.88¹ | 62.96 ± 2.14^{ns} | 0.719 ± 0.017^{ns} |
| knockout | | | |
| Mdr1ab | 44.51 ± 1.56¹ | 62.16 ± 2.05^{ns} | 0.757 ± 0.021^{ns} |
| knockout | | | |
| Bcrp; Mdr1ab | 56.46 ± 1.17^{1,2,3} | 64.47 ± 1.29^{ns} | 0.705 ± 0.014^{ns} |
| knockout | | | |

¹p < 0.01 relative to Wild-type mice
²p < 0.01 relative to Bcrp1 KO and Mdr1ab KO mice
³P < 0.001 relative to Wild-type mice
^{ns}Not significant relative to Wild-type mice.

TABLE 2
Pharmacokinetic parameters of temozolomide after oral
administration of 100 mg/kg.

| | AUC_{0-7 h, plasma} |
|---|---|
| Genotype | μg/ml · h |
| Wild-type | 154.2 ± 4.1 |
| Bcrp; Mdr1ab | 164.5 ± 4.5^{ns} |
| knockout | |

### REFERENCES

1. de Vries, N. A., Beijnen, J. H., Boogerd, W., and van Tellingen, O. 2006. Blood-brain barrier and chemotherapeutic treatment of brain tumors. Expert Rev Neurother. 6:1199-1209.

2. Wolburg, H. and Lippoldt, A. 2002. Tight junctions of the blood-brain barrier: development, composition and regulation. Vascul. Pharmacol. 38:323-337.

3. Schinkel, A. H. 1999. P-glycoprotein, a gatekeeper in the blood-brain barrier. Advan. Drug Delivery. Rev. 36:179-194.

4. Breedveld, P., Pluim, D., Cipriani, G., Wielinga, P., van Tellingen, O., Schinkel, A. H., and Schellens, J. H. 2005. The effect of Bcrp1 (Abcg2) on the in vivo pharmacokinetics and brain penetration of imatinib mesylate (Gleevec): implications for the use of breast cancer resistance protein and P-glycoprotein inhibitors to enable the brain penetration of imatinib in patients. Cancer Res. 65:2577-2582.

5. Stewart, L. A. 2002. Chemotherapy in adult high-grade glioma: a systematic review and meta-analysis of individual patient data from 12 randomised trials. Lancet 359:1011-1018.

6. Medical Research Council Brain Tumor Working Party. 2001. Randomized trial of procarbazine, lomustine, and vincristine in the adjuvant treatment of high-grade astrocytoma: a Medical Research Council trial. J Clin Oncol 19:509-518.

7. Yung, W. K., Prados, M. D., Yaya-Tur, R., Rosenfeld, S. S., Brada, M., Friedman, H. S., Albright, R., Olson, J., Chang, S. M., O'Neill, A. M. et al. 1999. Multicenter phase II trial of temozolomide in patients with anaplastic astrocytoma or anaplastic oligoastrocytoma at first relapse. Temodal Brain Tumor Group. J Clin Oncol 17:2762-2771.

8. Yung, W. K., Albright, R. E., Olson, J., Fredericks, R., Fink, K., Prados, M. D., Brada, M., Spence, A., Hohl, R. J., Shapiro, W. et al. 2000. A phase II study of temozolomide vs. procarbazine in patients with glioblastoma multiforme at first relapse. Br J Cancer 83:588-593.

9. Brada, M., Hoang-Xuan, K., Rampling, R., Dietrich, P. Y., Dirix, L. Y., Macdonald, D., Heimans, J. J., Zonnenberg, B. A., Bravo-Marques, J. M., Henriksson, R. et al. 2001. Multicenter phase II trial of temozolomide in patients with glioblastoma multiforme at first relapse. Ann Oncol 12:259-266.

10. Stupp, R., Mason, W. P., van den Bent, M. J., Weller, M., Fisher, B., Taphoorn, M. J., Belanger, K., Brandes, A. A., Marosi, C., Bogdahn, U. et al. 2005. Radiotherapy plus concomitant and adjuvant temozolomide for glioblastoma. N. Engl. J.Med. 352:987-996.

11. Kim, H., Likhari, P., Parker, D., Statkevich, P., Marco, A., Lin, C. C., and Nomeir, A. A. 2001. High-performance liquid chromatographic analysis and stability of anti-tumor agent temozolomide in human plasma. J Pharm Biomed Anal 24:461-468.

12. Schinkel, A. H., Wagenaar, E., van Deemter, L., Mol, C. A., and Borst, P. 1995. Absence of the mdrla P-Glycoprotein in mice affects tissue distribution and pharmacokinetics of dexamethasone, digoxin, and cyclosporin A. J. Clin. Invest. 96:1698-1705.

13. Jonker, J. W., Smit, J. W., Brinkhuis, R. F., Maliepaard, M., Beijnen, J. H., Schellens, J. H., and Schinkel, A. H. 2000. Role of Breast Cancer Resistance Protein in the Bioavailability and Fetal Penetration of Topotecan. J. Natl. Cancer Inst. 2000.Oct. 18, 1992(20):1651-1656. 92:1651-1656.

14. Kemper, E. M., van Zandbergen, A. E., Cleypool, C., Mos, H. A., Boogerd, W., Beijnen, J. H., and van Tellingen, O. 2003. Increased penetration of paclitaxel into the brain by inhibition of P-Glycoprotein. Clin. Cancer Res. 9:2849-2855.

15. Tsang,L. L., Quarterman, C. P., Gescher, A., and Slack, J. A. 1991. Comparison of the cytotoxicity in vitro of temozolomide and dacarbazine, prodrugs of 3-methyl-(triazen-1-yl)imidazole-4-carboxamide. Cancer Chemother Pharmacol 27:342-346.

16. Prados, M. D., Lamborn, K. R., Chang, S., Burton, E., Butowski, N., Malec, M., Kapadia, A., Rabbitt, J., Page, M. S., Fedoroff, A. et al. 2006. Phase 1 study of erlotinib HCl alone and combined with temozolomide in patients with stable or recurrent malignant glioma. Neuro-oncol 8:67-78.

17. Yang, C., Yang, C., Huang, C., Cheng, A., Whang-Peng, J., and Yang, P. 2006. Erlotinib completely reverses topotecan-resistance in multidrug resistant cancer cells overexpressing breast cancer resistant protein 1. AACR Meeting Abstracts 2006:1273-127c.

18. Peng, B., Dutreix, C., Mehring, G., Hayes, M. J., Ben Am, M., Seiberling, M., Pokorny, R., Capdeville, R., and Lloyd, P. 2004. Absolute bioavailability of imatinib (Glivec) orally versus intravenous infusion. J. Clin. Pharmacol. 44:158-162.

19. Dai, H., Marbach, P., Lemaire, M., Hayes, M., and Elmquist, W. F. 2003. Distribution of STI-571 to the Brain Is Limited by P-Glycoprotein-Mediated Efflux. J. Pharmacol. Exp. Ther. 304:1085-1092.

20. Mason, W. P. and Cairncross, J. G. 2005. Drug Insight: temozolomide as a treatment for malignant glioma—impact of a recent trial. Nat Clin Pract Neurol 1:88-95.

21. Schinkel, A. H., Mayer, U., Wagenaar, E., Mol, C. A. A. M., van Deemter, L., Smit, J. J. M., van der Valk, M. A., Voordouw, A. C., Spits, H., van Tellingen, O. et al. 1997. Normal viability and altered pharmacokinetics in mice lacking mdrl-type (drug-transporting) P-glycoproteins. Proc Natl Acad Sci USA 94:4028-4033.

22. Zhou, S., Schuetz, J. D., Bunting, K. D., Colapietro, A. M., Sampath, J., Morris, J. J., Lagutina, I., Grosveld, G. C., Osawa, M., Nakauchi, H. et al. 2001. The ABC transporter Bcrp1/ABCG2 is expressed in a wide variety of stem cells and is a molecular determinant of the side-population phenotype. Nat. Med. 7:1028-1034.

23. Kemper, E. M., van Zandbergen, A. E., Cleypool, C., Mos, H. A., Boogerd, W., Beijnen, J. H., and van Tellingen, O. Increased penetration of paclitaxel into the brain by inhibition of P-Glycoprotein. Clin. Cancer Res., 9: 2849-2855, 2003.

24. Oostendorp, R. L., Buckle, T., Beijnen, J. H., van, T. O., and Schellens, J. H. The effect of P-gp (Mdr1a/1b), BCRP (Bcrp1) and P-gp/BCRP inhibitors on the in vivo absorption, distribution, metabolism and excretion of imatinib. Invest New Drugs, 2008.

25. Wick, W., Steinbach, J. P., Kuker, W. M., Dichgans, J., Bamberg, M., and Weller, M. One week on/one week off: a novel active regimen of temozolomide for recurrent glioblastoma. Neurology, 62: 2113-2115, 2004.

1-40. (canceled)

41. A method of treating a patient, said method comprising administering a BCRP inhibitor, a P-gp inhibitor and a chemotherapeutic agent to the patient, wherein the chemotherapeutic agent is an imidazotetrazine.

42. The method of claim 41, wherein administration of the BCRP inhibitor and/or the P-gp inhibitor causes an increase in the amount of the chemotherapeutic agent in the brain of the patient being treated.

43. The method of claim 41, wherein said method is for treating cancer in a patient.

44. The method of claim 41 wherein the BCRP inhibitor and/or the P-gp inhibitor is for reducing transport of the chemotherapeutic agent across the blood brain barrier from the brain into the blood of the patient being treated.

45. The method of claim 41 wherein the chemotherapeutic agent is temozolomide.

46. The method of claim 41 wherein the BCRP inhibitor is selected from the group consisting of: erlotinib, pantoprazole, tryprostatin A, fumitremorgin C, demethoxy-fumitremorgin C analogs, Kol32, Kol34, Kol43, GF120918 (elacridar), CI1033, estrone, 17 beta-estradiol, estradiol-17-beta-D-glucuronide, iressa (gefitinib or ZD 1839), imatinib mesylate (STI571 or Gleevec), EKI-785, Novobiocin, diethylstilbestrol, Tamoxifen, TAG-11, TAGO139, reserpine, VX710 (Biricodar or Incel), Tryprostatin A, Flavonoids (chrysin and biochanin A), Ritonavir, Saquinavir, Nelfinavir, Omeprazole, Cyclosporine A, XR9051 and XR9576 (tariquidar).

47. The method of claim 46 wherein the chemotherapeutic agent is temozolomide.

48. The method of claim 41 wherein the P-gp inhibitor is selected from the group consisting of: cyclosporine A, verapamil, [3′-desoxy-3′-oxo-MeBmt]¹-Ciclosporin, [3′-desoxy-3′-oxo-MeBmt]¹-[Val]²-Ciclosporin (PSC388), [3′-desoxy-3′-oxo-MeBmt]¹-[Nva]²-Ciclosporin, Cyclo-[Pec-MeVal-Val-MeAsp(β-O-t-Bu)-MeIle-MeIle-Gly-MeVal-Tyr(Me)-L-Lact], Cyclo-[Pec-MeVal-Val-MeAsp-MeIle-MeIle-Gly-MeVal-Tyr(Me)-D-Lact], GF120918 (elacridar), MS-209, XR-9576, VX-710, R-101933, NSC-38721, OC-144093, LY-335979, XR9051 and XR9576 (tariquidar).

49. The method of claim 48 wherein the chemotherapeutic agent is temozolomide.

50. The method of claim 41 wherein the BCRP inhibitor is also a P-gp inhibitor.

51. The method of claim 41 wherein the BCRP inhibitor and/or the P-gp inhibitor is GF120918 (elacridar).

52. The method of claim 41 wherein the BCRP inhibitor is erlotinib.

53. The method of claim 43 wherein the cancer is a tumour that is sensitive to temozolomide.

54. The method of claim 43 wherein the cancer is glioma or glioblastoma multiforme (GBM).

55. A method of treating a patient having cancer of the central nervous system, the method comprising administering to the patient a therapeutically effective amount of elacridar, and a therapeutically effective amount of temozolomide.

56. A pharmaceutical composition comprising a BCRP inhibitor, a P-gp inhibitor and a chemotherapeutic agent, wherein the chemotherapeutic agent is an imidazotetrazine.

57. A pharmaceutical composition comprising temozolomide and elacridar.

58. A method of treating a patient, said method comprising administering a BCRP inhibitor and a chemotherapeutic agent to the patient, wherein administration of the BCRP inhibitor causes an increase in the amount of the chemotherapeutic agent in the brain of the patient being treated, and wherein the chemotherapeutic agent is an imidazotetrazine.

59. The method of claim 58, wherein said method is for treating cancer in a patient.

60. The method of claim 59, wherein administration of the BCRP inhibitor and/or the P-gp inhibitor causes a reduction in transport of the chemotherapeutic agent across the blood brain barrier from the brain into the blood of the patient.

61. A method of treating a patient, said method comprising administering a P-gp inhibitor and a chemotherapeutic agent to the patient, wherein administration of the P-gp inhibitor causes an increase in the amount of the chemotherapeutic agent in the brain of the patient being treated, and wherein the chemotherapeutic agent is an imidazotetrazine.

62. The method of claim 61, wherein said method is for treating cancer in a patient.

63. The method of claim 62, wherein administration of the BCRP inhibitor and/or the P-gp inhibitor causes a reduction in transport of the chemotherapeutic agent across the blood brain barrier from the brain into the blood of the patient.

64. A kit of parts comprising a packaging having a BCRP inhibitor, a P-gp inhibitor, and a chemotherapeutic agent, wherein the chemotherapeutic agent is an imidazotetrazine.

65. A kit of parts comprising a packaging having a BCRP inhibitor and a chemotherapeutic agent, wherein the kit is for use in the treatment of cancer, wherein the BCRP inhibitor is for increasing the amount of the chemotherapeutic agent in the brain of a patient being treated, and wherein the chemotherapeutic agent is an imidazotetrazine.

66. A kit of parts comprising a packaging having a P-gp inhibitor and a chemotherapeutic agent, wherein the kit is for use in the treatment of cancer, wherein the P-gp inhibitor is for increasing the amount of the chemotherapeutic agent in the brain of a patient being treated, and wherein the chemotherapeutic agent is an imidazotetrazine.
